(19) United States
(12) Patent Application Publication
(10) Pub. No.: US 2008/0193967 A1
(43) Pub. Date: Aug. 14, 2008

(54) Colorimetric Sensor Constructed Of Diacetylene Materials

(76) Inventors: G. Marco Bommarito, Stillwater, MN (US); Brinda B. Lakshmi, Woodbury, MN (US)

Correspondence Address: 3M INNOVATIVE PROPERTIES COMPANY, PO BOX 33427, ST. PAUL, MN, 55133-3427, US

(21) Appl. No.: 11/721,689

(22) PCT Filed: Dec. 16, 2005
(86) PCT No.: PCT/US05/45607

§ 371 Date: Jun. 14, 2007

Related U.S. Application Data
(60) Provisional application No. 60/636,993, filed on Dec. 17, 2004.

(51) Int. Cl. C12Q 1/04; B01J 19/00; G01N 21/78

(52) U.S. Cl. 435/34; 422/82.05; 436/164

(57) ABSTRACT

Colorimetric sensors for detection of an analyte are disclosed. Methods of using the colorimetric sensor and a kit for the colorimetric detection of an analyte are also disclosed.

### CROSS-REFERENCE TO RELATED APPLICATIONS

The present application claims priority to U.S. Provisional Patent Application Ser. No. 60/636,993, filed on Dec. 17, 2004, which is incorporated herein by reference in its entirety.

### BACKGROUND

Current techniques for the detection of microbes, particularly bacteria resistant to antibiotics, are generally time consuming and typically involve culturing the bacteria in pure form. One such microbe of significant interest is Staphylococcus aureus (“S. aureus”), which is a pathogen causing a wide spectrum of infections including: superficial lesions such as small skin abscesses and wound infections; systemic and life threatening conditions such as endocarditis, pneumonia and septicemia; as well as toxinoses such as food poisoning and toxic shock syndrome. S. aureus is resistant to all but a few select antibiotics.

Analysis of microbes using a wide variety of conventional techniques have been attempted. For example, methods include the use of fluorometric immunochromatography (e.g., rapid analyte measurement procedure such as that described in U.S. Pat. No. 5,753,517), ELISA (e.g., calorimetric ELISA), and other calorimetric techniques. Colorimetric sensors that include polydiacetylene (PDA) materials are described in U.S. Pat. No. 5,622,872 and Publication WO 02/00920; U.S. Pat. Nos. 6,395,561 B1; 6,306,598 B1; 6,277,652; 6,183,722; and 6,080,423.

Diacetylenes are typically colorless as monomers in solution, and undergo addition polymerization, either thermally or by actinic radiation. As the polymerization proceeds, these compounds undergo a contrasting color change to blue or purple. When exposed to external stimuli such as heat, physical stress, or a change of solvents or counterions, polydiacetylenes exhibit a further color change produced by distortion of the planar backbone conformation. For example, polydiacetylene assemblies are known to change color from blue to red with an increase in temperature or changes in pH due to conformational changes in the conjugated backbone as described in Mino, et al., Langmuir, Vol. 8, p. 594, 1992; Chance, et al., Journal of Chemistry and Physics, Vol. 71, 206, 1979; Shibutag, Thin Solid Films, Vol. 179, p. 433, 1989; Kaneko, et al. Thin Solid Films, Vol. 210, 548, 1992; and U.S. Pat. No. 5,672,465.

Although methods of detecting S. aureus, as well as other microbes, have been described in the art, there would be advantage in improved methods of detection.

### SUMMARY

The present invention provides a colorimetric sensor to detect the presence of analytes by spectral changes (color changes visible to the naked eye or with a calorimeter) that occur as a result of the interaction of the analytes in a manner that cause conformational changes to polydiacetylene assemblies. The polydiacetylene assemblies indicate the presence of an analyte in a simple yet highly sensitive manner. A calorimetric system for detecting an analyte is provided, comprising a colorimetric sensor comprising a receptor; a polymerized composition comprising at least one diacetylene compound (by this it is meant that the polymerized composition is formed from polymerization of the diacetylene compound); wherein the receptor is incorporated into the polymerized composition to form a transducer; and a buffer composition that mediates the interaction between the analyte and the transducer, wherein the buffer system includes two or more different buffers; wherein the transducer exhibits a color change when contacted with an analyte.

In one embodiment, the buffer composition is a combination of a higher ionic strength buffer with a lower ionic strength buffer. In a preferred embodiment, the buffer composition is selected from the group consisting of HEPES buffer, Imidazole buffer, PBS buffer, and combinations thereof. In one embodiment, the buffer mediates the interaction of the analyte by ionic interactions with the transducer. In another embodiment, the buffer composition mediates the interaction of the analyte by enhancing hydrophobic interactions with the transducer. The transducer may be dispersed in an aqueous solution or coated on a substrate.

In another embodiment, the calorimetric system further comprises a probe. In a preferred embodiment, the probe is selected from the group consisting of fibrinogen, streptavidin, IgG, and combinations thereof.

In another embodiment, the calorimetric system further comprises a surfactant. In a preferred embodiment, the surfactant comprises a nonionic surfactant.

In an exemplary embodiment, the transducer of the colorimetric system is a liposome and/or exhibits a color change upon contact with the buffer composition.

In an exemplary embodiment, the diacetylene compound (i.e., the starting material for the polydiacetylene material) is of the formula

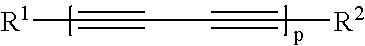

wherein R¹ comprises
C₁-C₂₀ alkyl,

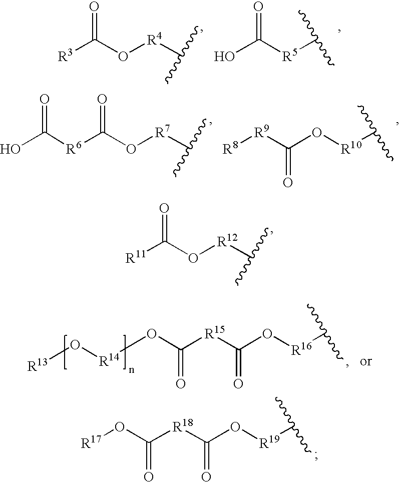

R² comprises

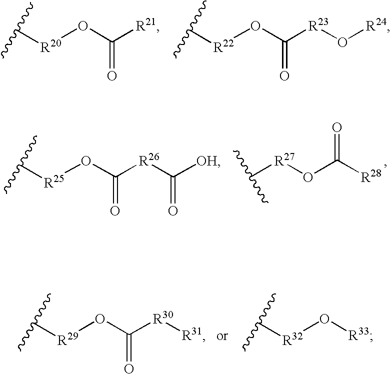

wherein R³, R⁸, R¹³, R²¹, R²⁴, R³¹, and R³³ are independently C₁-C₂₀ alkyl; R⁴, R⁵, R⁷, R¹⁴, R¹⁶, R¹⁹, R²⁰, R²², R²⁵, and R³² are independently C₁-C₁₄ alkylene; R⁶, R¹⁵, R¹⁸, and R²⁶ are independently C₁-C₁₄ alkylene, C₂-C₈ alkenylene, or C₆-C₁₃ arylene; R⁹ is C₁-C₁₄ alkylene or —NR³⁴—; R¹⁰, R¹², R²⁷, and R²⁹ are independently C₁-C₁₄ alkylene or (C₁-C₁₄ alkylene)-(C₂-C₈ arylene); R¹¹ and R²⁸ are independently C₂-C₃₀ alkynyl; R¹⁷ is an ester-activating group; R²³ is C₆-C₁₃ arylene; R³⁰ is C₁-C₁₄ alkylene or —NR³⁶—; R³⁴ and R³⁶ are C₁-C₄ alkyl; p is 1-5 (herein, “diacetylene” is used to encompass compounds with two to ten C—C triple bonds); and n is 1-20; wherein R¹ and R² are not the same.

In one embodiment, the receptor in the colorimetric system comprises a phospholipid selected from the group consisting of phosphocholines, phosphoethanolamines, phosphatidylethanolamines, phosphatidylserines, phosphatidylglycerols, and combinations thereof.

A method for the detection of an analyte is also provided. The method includes forming a colorimetric sensor comprising a receptor and a polymerized composition comprising a diacetylene (i.e., the polymerized composition is derived from polymerization of the diacetylene), wherein the receptor is incorporated into the polymerized composition to form a transducer capable of exhibiting a color change; contacting the sensor with a probe; contacting the sensor with a sample suspected of containing a target analyte in the presence of a buffer composition comprising two or more different buffers; and observing a color change if the analyte is present.

In another embodiment, a method for the detection of an analyte is provided, comprising forming a colorimetric sensor, comprising a receptor and a polymerized composition comprising a diacetylene, wherein the receptor is incorporated into the polymerized composition to form a transducer capable of exhibiting a color change in the presence of a probe; contacting the transducer with a sample suspected of containing a target analyte, and a probe that has an affinity for both the target analyte and the receptor in the presence of a buffer composition comprising two or more different buffers; and observing essentially no color change if the analyte is present. Preferably, the probe and sample suspected for containing a target analyte may be combined to form a mixture before contacting the transducer.

In an exemplary embodiment, the analyte is selected from the group consisting of S. aureus, protein A, PBP2′, E. coli, and Pseudomonas aeruginosa. In most embodiments, the colorimetric system exhibits an observable color change within 60 minutes of contacting the transducer with an analyte.

### DEFINITIONS

For the following defined terms, these definitions shall be applied, unless a different definition is given in the claims or elsewhere in this specification:

As used herein, the term “alkyl” refers to a straight or branched chain or cyclic monovalent hydrocarbon group having a specified number of carbon atoms. Alkyl groups include those with one to twenty carbon atoms. Examples of “alkyl” as used herein include, but are not limited to, methyl, ethyl, n-propyl, n-butyl, n-pentyl, isobutyl, and isopropyl, and the like. It is to be understood that where cyclic moieties are intended, at least three carbons in said alkyl must be present. Such cyclic moieties include cyclopropyl, cyclobutyl, cyclopentyl, cyclohexyl, and cycloheptyl.

As used herein, the term “alkylene” refers to a straight or branched chain or cyclic divalent hydrocarbon group having a specified number of carbon atoms. Alkylene groups include those with one to fourteen carbon atoms. Examples of “allcylene” as used herein include, but are not limited to, methylene, ethylene, trimethylene, tetramethylene and the like. It is to be understood that where cyclic moieties are intended, at least three carbons in said alkylene must be present. Such cyclic moieties include cyclopropylene, cyclobutylene, cyclopentylene, cyclohexylene, and cycloheptylene.

As used herein, the term “alkenylene” refers to a straight or branched chain or cyclic divalent hydrocarbon group having a specified number of carbon atoms and one or more carbon—carbon double bonds. Alkenylene groups include those with two to eight carbon atoms. Examples of “alkenylene” as used herein include, but are not limited to, ethene-1,2-diyl, propene-1,3-diyl, and the like.

As used herein, the term “arylene” refers to divalent unsaturated aromatic carboxylic groups having a single ring, such as phenylene, or multiple condensed rings, such as naphthylene or anthrylene. Arylene groups include those with six to thirteen carbon atoms. Examples of “arylene” as used herein include, but are not limited to, benzene-1,2-diyl, benzene-1,3-diyl, benzene-1,4-diyl, naphthalene-1,8-diyl, and the like.

As used herein, the term “alkylene-arylene,” refers to an alkylene moiety as defined above bonded to an arylene moiety as defined above. Examples of “alkylene-arylene” as used herein include, but are not limited to, —CH₂-phenylene, —CH₂CH₂-phenylene, and —CH₂CH₂CH₂-phenylene.

As used herein, the term “alkynyl” refers to a straight or branched chain or cyclic monovalent hydrocarbon group having from two to thirty carbons and at least one carbon-carbon triple bond. Examples of “alkynyl” as used herein include, but are not limited to, ethynyl, propynyl and butynyl.

As used herein, the term “analyte(s)” refers to any material that can be detected by the sensor system of the present invention. Such materials include, but are not limited to, small molecules, pathogenic and non-pathogenic organisms, toxins, membrane receptors and fragments, volatile organic compounds, enzymes and enzyme substrates, antibodies, antigens, proteins, peptides, nucleic acids, and peptide nucleic acids. “Target analyte” refers to the material targeted for detection in a sensor system.

As used herein, the term “bacteria” refers to all forms of microorganisms considered to be bacteria including cocci, bacilli, spirochetes, sheroplasts, protoplasts, etc.

As used herein, the term “receptor” refers to any molecule or assembly of molecules with an affinity for a target analyte and/or a probe. Receptor includes, but is not limited to, naturally occurring or synthetic receptors such as lipids, surface membrane proteins, enzymes, lectins, antibodies, recombinant proteins, synthetic proteins, nucleic acids, c-glycosides, carbohydrates, gangliosides, and chelating agents.

As used herein, the terms “assembly,” or “self-assembly,” refers to any self-ordering of diacetylene molecules and phospholipids prior to polymerization. See J. Israelachvili, Intermolecular and Surface Forces(2^{nd} Ed.), Academic Press, New York (1992), pp. 321-427.

As used herein, the term “self-assembling monolayer(s)” (SAMs) refers to any ordered ultrathin organic film formed on a given substrate by spontaneous self-ordering. A. Ulman, An Introduction to Ultrathin Organic Films, Academic Press, New York (1991), pp. 237-301.

As used herein, the term “transducer” describes a material capable of turning a recognition event such as a covalent bond or a noncovalent interaction (e.g., electrostatic interaction, polar interaction, van der Waals forces) at the molecular level into an observable signal.

“Probe” refers to a constituent that is capable of interacting with the target analyte and/or the receptor. Accordingly, the probe is a type of “detectable binding reagent” i.e., an agent that specifically recognizes and interacts or binds with an analyte (i.e., the target analyte) and/or the receptor, wherein the probe has a property permitting detection when bound. “Specifically interact” means that detectable binding agent physically interacts with the target analyte or receptor to the substantial exclusion of other analytes also present in the sample. The binding of a detectable binding reagent useful according to the invention has stability permitting the measurement of the binding.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF ILLUSTRATIVE EMBODIMENTS

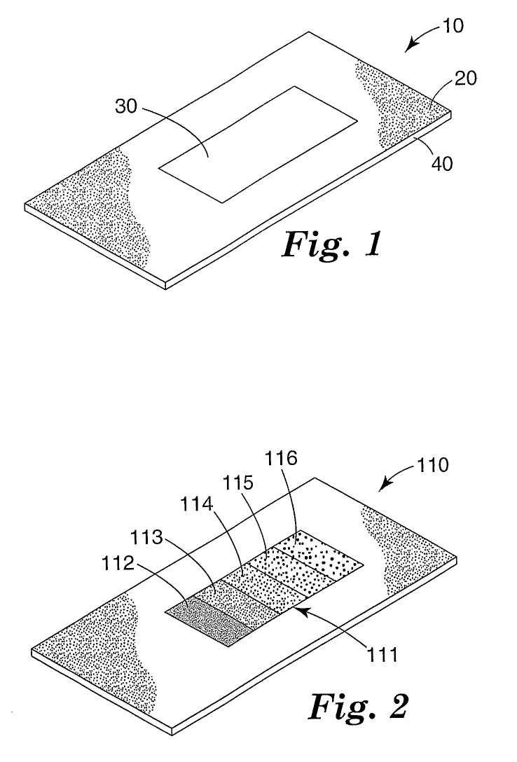

FIG. 1 shows a schematic representation of a calorimetric sensor of the present invention.

FIG. 2 shows a schematic representation of a calorimetric sensor array of the present invention.

### DETAILED DESCRIPTION OF ILLUSTRATIVE EMBODIMENTS

The present invention provides a calorimetric sensor system for detection of an analyte. The calorimetric system includes a calorimetric sensor comprising a receptor and a polymerized diacetylene material (polydiacetylene assemblies, which refer to an organized polydiacetylene structure that may (but not necessarily) include other components), wherein the receptor is incorporated within the polydiacetylene to form a transducer capable of providing a color change upon binding with a probe and/or analyte. The colorimetric sensor can function in solution or coated on a substrate.

### Polydiacetylene Assemblies

The diacetylene compounds of the present invention can self assemble in solution to form ordered assemblies that can be polymerized using any actinic radiation such as, for example, electromagnetic radiation in the UV or visible range of the electromagnetic spectrum. Polymerization of the diacetylene compounds result in polymerization reaction products that have a color in the visible spectrum less than 570 nanometers (nm), between 570 nm and 600 nm (including the endpoints), or greater than 600 nm, depending on their conformation and exposure to external factors. Typically, polymerization of the diacetylene compounds disclosed herein result in meta-stable blue phase polymer networks that include a polydiacetylene backbone. These meta-stable blue phase polymer networks undergo a color change from bluish to reddish-orange upon exposure to external factors such as heat, a change in solvent or counterion, if available, or physical stress, for example.

The ability of the diacetylene compounds and their polymerization products disclosed herein to undergo a visible color change upon exposure to physical stress make them candidates for the preparation of sensing devices for detection of an analyte. The polydiacetylene assemblies formed from the disclosed diacetylene compounds can function as a transducer in biosensing applications.

The structural requirements of a diacetylenic molecule for a given sensing application are typically application specific. Features such as overall chain length, solubility, polarity, crystallinity, and presence of functional groups for further molecular modification all cooperatively determine a diacetylenic molecule's ability to serve as a useful sensing material. For example, in the case of biodetection of an analyte in aqueous media, the structure of the diacetylenic compound should be capable of forming a stable dispersion in water, polymerizing efficiently to a colored material, incorporating appropriate receptor chemistry for binding to an analyte, and transducing that binding interaction by means of a color change. These abilities are dependent on the structural features of the diacetylene compounds.

The diacetylene compounds of the present invention possess the capabilities described above and can be easily and efficiently polymerized into polydiacetylene assemblies that undergo the desired color changes. Additionally, the diacetylene compounds allow for the incorporation of large excesses of unpolymerizable material, such as a receptor described below, while still forming a stable, polymerizable solution.

The disclosed diacetylene compounds (the starting material) can be synthesized in a rapid high-yielding fashion, including high-throughput methods of synthesis. The presence of functionality in the backbones of the diacetylenic compounds (the starting material) such as heteroatoms for example, provides for the possibility of easy structural elaboration in order to meet the requirements of a given sensing application. The diacetylenic compounds can be polymerized into the desired polydiacetylene backbone containing network by adding the diacetylene to a suitable solvent, such as water for example, sonicating the mixture, and then irradiating the solution with ultraviolet light, typically at a wavelength of 254 nm. Upon polymerization the solution undergoes a color change to bluish-purple.

Diacetylenes (the starting material) useful in the present invention typically contain an average carbon chain length of at least 8 with at least one functional group such as a carboxyl group, primary or tertiaty amine groups, methyl esters of carboxyl, etc. Suitable diacetylenes include those described in U.S. Pat. No. 5,491,097 (Ribi et al.), PCT Publication No. WO 02/00920, U.S. Pat. No. 6,306,598, and PCT Publication WO 01/71317.

In a preferred embodiment, the polydiacetylene assemblies include polymerized compounds resulting from the diacetylenes of the formula

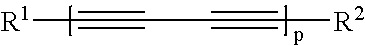

where R¹ is

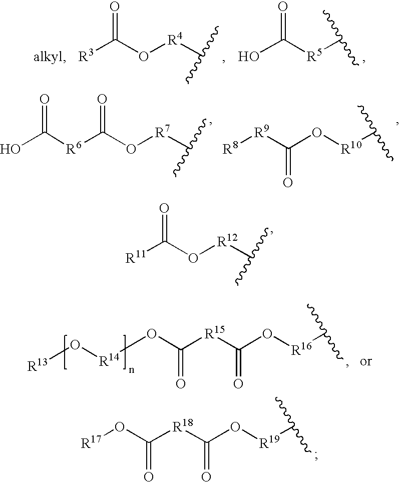

R² is

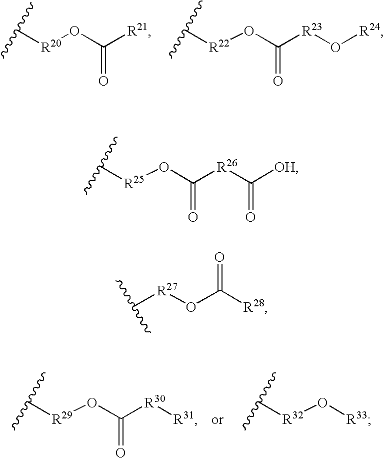

R³, R⁸, R¹³, R²¹, R²⁴, R³¹, and R³³ are independently alkyl; R⁴, R⁵, R⁷, R¹⁴, R¹⁶, R¹⁹, R²⁰, R²², R²⁵, and R³² are independently alkylene; R⁶, R⁵, R¹⁸, and R²⁶ are independently alkylene, alkenylene, or arylene; R⁹ is alkylene or —NR³⁴—; R¹⁰, R¹², R²⁷, and R²⁹ are independently alkylene or alkylene-arylene; R¹ and R²⁸ are independently alkynyl; R¹⁷ is an ester-activating group; R²³ is arylene; R³⁰ is alkylene or —NR³⁶—; R³⁴ and R³⁶ are independently H or C₁-C₄ alkyl; p is 1-5; and n is 1-20; where R¹ and R² are not the same.

Exemplary compounds are further described in U.S. Publication No. 2005/0101794-A1 and U.S. Publication Nos. 2004/0126897-A1 and 2004/0132217-A1.

In a preferred embodiment, R¹ is

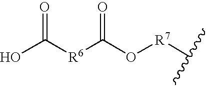

wherein R⁷ is ethylene, trimethylene, tetramethylene, pentamethylene, hexamethylene, heptamethylene, octamethylene, or nonamethylene, and R⁶ is ethylene, trimethylene, ethenylene, or phenylene; and wherein R² is

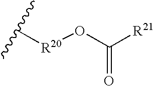

wherein R²⁰ is ethylene, trimethylene, tetramethylene, pentamethylene, hexamethylene, heptamethylene, octamethylene, or nonamethylene, and wherein R²¹ is undecyl, tridecyl, pentadecyl, heptadecyl; and wherein p is 1.

The invention is inclusive of the compounds described herein including isomers, such as structural isomers and geometric isomers, salts, solvates, polymorphs, and the like.

Diacetylenes of the Formula XXIII can be prepared as outlined in Scheme 1 where n is typically 1 to 4 and m is typically 10 to 14.

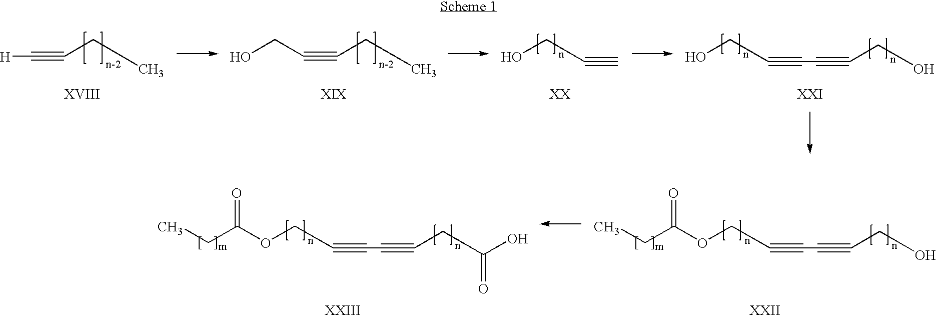

Compounds of formula XXIII can be prepared via oxidation from compounds of formula XXII by reaction with a suitable oxidizing agent in a suitable solvent such as DMF, for example. Suitable oxidizing agents include Jones reagent and pyridinium dichromate, for example. The aforesaid reaction is typically run for a period of time from 1 hour to 48 hours, generally 8 hours, at a temperature from 0° C. to 40° C., generally from 0° C. to 25° C.

Compounds of formula XXII can be prepared from compounds of formula XXI by reaction with a suitable acid chloride. Suitable acid chlorides include any acid chloride that affords the desired product such as lauroyl chloride, 1-dodecanoyl chloride, 1-tetradecanoyl chloride, 1-hexadecanoyl chloride, and 1-octadecanoyl chloride for example. Suitable solvents include ether, tetrahydrofuran, dichloromethane, and chloroform, for example. The aforesaid reaction is typically run for a period of time from 1 hour to 24 hours, generally 3 hours, at a temperature from 0° C. to 40° C., generally from 0° C. to 25° C., in the presence of a base such as trialkylamine or pyridine base.

Compounds of formula XXI are either commercially available (e.g. where n is 1-4) or can be prepared from compounds of the formula XVIII via compounds XIX and XX as outlined in Scheme 1 and disclosed in Abrams, Suzanne R.; Shaw, Angela C. “Triple-bond isomerizations: 2- to 9-decyn-1-ol,” Org. Synth. (1988), 66, 127-31 and Brandsma, L. “Preparative Acetylenic Chemistry,” (Elsevier Pub. Co., New York, 1971), for example.

Diacetylenic compounds as disclosed herein can also be prepared by reacting compounds of formula XXII with an anhydride such as succinic, glutaric, or phthalic anhydride in the presence of a suitable solvent, such as toluene. The aforesaid reaction is typically run for a period of time from 1 hour to 24 hours, generally 15 hours, at a temperature from 50° C. to 125° C., generally from 100° C. to 125° C.

The colorimetric sensors comprising the polymerized diacetylenes can serve as the basis for the colorimetric detection of a molecular recognition event. The sensor can be prepared by adding a receptor to the diacetylene monomers either prior to or after polymerization. The receptor is capable of functionalizing the polydiacetylene assemblies through a variety of means including physical mixing, covalent bonding, and noncovalent interactions (such as electrostatic interactions, polar interactions, etc).

Upon polymerization or thereafter, the receptor is effectively incorporated with the polymer network such that interaction of the receptor with an analyte results in a visible color change due to the perturbation of the conjugated ene-yne polymer backbone.

The incorporation of the receptor with the polydiacetylene assembly provides a structural shape capable of deformation in response to interaction or binding with a probe and/or analyte. Particularly useful receptors are assemblies of amphiphilic molecules with typically a rod shape molecular architecture that can be characterized by a packing parameter defined as: v/(a₀1_{c}) (Israelachvili, J. N. et al.; Q. Rev. Biophys.; 13, 121, 1980), where v is the volume taken up by the hydrocarbon components of the molecules (for example, the hydrocarbon chains of a phospholipid or a fatty acid), a₀ is the effective area taken up by the polar headgroup (for example the phosphate headgroup of a phospholipid or the carboxylic acid headgroup of a fatty acid), and 1_{c} is the so-called critical length, and generally describes the length of the molecule at the temperature of its environment. Preferred amphiphilic molecules for a receptor are those with packing parameter v/(a₀1_{c}) values between ⅓ and 1.

Examples of useful receptors include, but are not limited to, lipids, surface membrane proteins, enzymes, lectins, antibodies, recombinant proteins, synthetic proteins, nucleic acids, c-glycosides, carbohydrates, gangliosides, and chelating agents. In most embodiments, the receptor is a phospholipid. Suitable phospholipids include phosphocholines (e.g., 1,2-dimeristoyl-sn-glycero-3-phosphocholine), phosphoethanolamines, and phosphatidylethanolamines, phosphatidylserines, and phosphatidylglycerols such as those described in Silver, Brian L., The Physical Chemistry of Membranes, Chapter 1, pp 1-24 (1985).

In one embodiment, the receptor is physically mixed and dispersed among the polydiacetylene to form a structure wherein the structure itself has a binding affinity for the probe and/or analyte of interest. Structures include, but are not limited to, liposomes, micelles, and lamellas. In a preferred embodiment, the structure is a liposome. While not intending to be bound by theory, it is believed that the phospholipid mimics a cell membrane while the polydiacetylene assemblies allow the physico-chemical changes occurring to the liposomes to be translated into a visible color change. The liposomes as prepared possess a well-defined morphology, size distribution, and other physical characteristics such as a well-defined surface potential.

The ratio of receptor to diacetylene compounds (starting material) in the liposome can be varied based on the selection of materials and the desired colorimetric response. In most embodiments, the ratio of phospholipids to diacetylene compound (starting material) will be at least 25:75, and more preferably at least 40:60. In a preferred embodiment, the liposomes are composed of the diacetylene compound: HO(O)C(CH₂)₂C(O)O(CH₂)₄C≡C—C≡C(CH₂)₄O(O)C(CH₂)₁₂CH₃ [succinic acid mono-(12-tetradecanoyloxy-dodeca-5,7-diynyl) ester], and the zwitterionic phospholipid 1,2-dimeristoyl-sn-glycero-3-phosphocholine [DMPC] mixed in a 6:4 ratio.

Herein, the discussion of the PDA systems is directed to the use of liposomes in the receptor assembly; however, this discussion also applies to other receptor assemblies, including, for example, other planar configurations.

The liposomes are prepared by probe sonication of the material mixture suspended in a buffer solution that is referred to as the preparation buffer. For example, the preparation buffer can be a low ionic strength (5 mM) N-2-Hydroxyethylpiperazine-N′-2-ethanesulfonic acid [HEPES] buffer (pH=7.2). Another useful preparation buffer is a low ionic strength (2 mM) Tris Hydroxymethylaminoethane [TRIS] buffer (pH=8.5).

The colorimetric system of the present invention is designed to exploit the way a probe can interact with the liposomes containing both a receptor, such as a phospholipids, and the polymerized diacetylenes. The liposomes can be used as models for biological membranes that interact with a probe, such as a protein, as described in Oellerich, S. et al.; J. Phys. Chem. B; 2004, 108, 3871-3878; and Zuckermann, M. J.; Heimburg T.; Biophysi. J.; 2001, 81, 2458-2472. In general, at high lipid to protein concentration ratios, proteins will adsorb to the surface of the liposomes primarily through electrostatic interactions.

As the protein concentration is increased, and the lipid to protein concentration ratio is lowered, proteins continue to adsorb electrostatically to the surface of a liposome until they completely saturate or envelop the liposomes. As this process proceeds, both liposomes and the proteins can undergo morphological and conformational changes, until the hydrophobic segment of the proteins covering the liposome surface can begin to interact with the hydrophobic interior of the liposome structure. At this point, the proteins can become hydrophobically bound and penetrate the liposome structure, resulting in substantial morphological change in the liposome structure, with the size and permeability of the liposomes changing drastically. Eventually, the layers of proteins can result in the loss of suspension stability, flocculation, and finally, precipitation.

The presence of these electrostatic interactions is highly dependent not only on the type of proteins and lipids present but on their environment as well. Although not desiring to be bound by theory, it is believed that the ionic strength of a given buffer system would be helpful in establishing the surface potential of both liposomes and charged proteins, and thus their ability to interact significantly electrostatically.

For example, in a buffer system of low ionic strength (2-5 mM) at neutral pH (e.g., HEPES, TRIS), a charged probe can electrostatically adsorb to the polydiacetylene liposomes. Although the initial adsorption may not in itself trigger a substantial change in the size and morphology of the liposome, and thus an initially small or negligible colorimetric response, if the probe is present in excess relative to the lipid, it is likely that the probe will eventually become hydrophobically bound to the liposome and penetrate its interior membrane structure. At this point, one would expect that the large mechanical stresses imparted by the incorporation of the probe within the liposome structure would significantly change the polydiacetylene conformation, resulting in a concomitant calorimetric response readily observable.

Alternatively, if the probe is negatively-charged at neutral pH its capacity to interact electrostatically with the polydiacetylene liposomes is severely hindered, and the ability to generate a calorimetric response due to a hydrophobic interaction between probe and the receptor-containing polydiacetylene liposomes may be compromised. In this event, using a high ionic strength buffer (>100 mM) at neutral pH (e.g., phosphate buffer saline (PBS), Imidazole buffer) would provide a means to decrease the surface potential of the liposomes (by screening the surface charge of the liposome), facilitating the direct hydrophobic interaction of non-charged probes with the liposomes, and resulting in the incorporation of that protein within the structure of the liposome. Thus, in this case, the buffer system assists in enabling a substantial colorimetric response, which would otherwise not take place. Although the higher ionic strength of the buffer, because of its effect on the surface potential of the liposomes, can introduce a significant calorimetric response in the absence of a probe, it has been determined that when the probe is present, the calorimetric response is significantly enhanced due to the protein-liposome hydrophobic interactions. This result has very useful practical consequences: the detection time at a given limit of detection can be significantly shortened, or conversely, for a fixed assay time the limit of detection can be significantly lowered.

Based on this phenomena, the probe can be selected based on its ability to interact specifically with both a given analyte target and the polydiacetylene liposome to trigger a colorimetric response. The calorimetric response of the polydiacteylene-containing liposome is directly proportional to the concentration of the probe or a probe-analyte complex in those cases of direct analysis.

The selection of probe for a particular application will depend in part on the probes' size, shape, charge, hydrophobicity, and affinity towards molecules. The probes may be positively charged, negatively charged, or zwitterionic depending on the pH of the environment. At a pH below the isoelectric point of a probe, the probe is positively charged and above this point it is negatively charged. As used herein, the term “isoelectric point” refers to the pH at which the probe has a net charge of zero.

In order to design a biochemical assay with a polydiacetylene/phospholipid system, knowing the isoelectric point of the receptor (or probe) will affect the choice of buffer combinations. A probe with lower isoelectric point may require higher ionic strength buffers to obtain a change in morphology of the liposome. A higher isoelectric point protein can be used in low ionic strength buffer like HEPES buffer to produce a color change.

Given this general mechanism it is important to define detection assays taking into consideration not only the polydiacetylene liposome composition (e.g., choice of the phospholipid being used and the ratio of phospholipid to diacetylene), and the probe being used (e.g., polymixin, fibrinogen, antibodies), but also the aqueous environment established by the choice of a buffer system.

The buffer composition of the present invention provides a system capable of resisting changes in pH in the presence of other components, consisting of a conjugate acid-base pair in which the ratio of proton acceptor to proton donor is near unity. In addition, the buffer compositions of the present invention mediate the physical or chemical interaction between the analyte and the components of the colorimetric sensor. For example, in one embodiment, the buffer composition inhibits the interaction of the analyte with the receptor. In another embodiment, the buffer composition facilitates the interaction of the analyte with the receptor. Buffer compositions that may be particularly useful include HEPES buffer, Imidazole buffer, and PBS buffer.

In a preferred embodiment, a combination of buffers (i.e., different buffers) is used to adjust the appropriate ionic strength for a given application based on the selection of the probe and/or the target analyte to be detected.

Combining two or more different buffers is a convenient means of tailoring the physical properties of the buffer system to achieve the appropriate balance of electrostatic and hydrophobic components in the liposome-protein probe interaction.

For example, in a system containing only HEPES buffer, which has a pH of 7.2, polymyxin (with an isoelectric point of 7.7) has a positive charge and readily adheres to the negatively charged polar head group of a phospholipids, and can induce a color change from blue to red in the colorimetric sensor. Fibrinogen, with an isoelectric point of 5.3, has a negative charge in the same HEPES buffer composition, which prevents adsorption or any electrostatic interaction with the polar head group of the phospholipids.

Alternatively, in the presence of the buffers with higher ionic strength, such as imidazole or PBS, the ionic strength alters the morphology of the liposome (or other transducer structure), to expose the hydrophobic portions. In calorimetric systems containing the higher ionic strength buffer compositions, fibrinogen contains hydrophobic parts in the structure that interacts with the phospholipids to cause a color change.

One convenient method of achieving the optimum balance of electrostatic and hydrophobic components in the liposome-protein interaction is to use a mixture of two or more different buffers. For example, mixing a low ionic strength organic buffer (HEPES, Tris) with an inorganic buffer (PBS) at a different ionic strength, can allow one to span the range of buffer properties bracketed by the single buffer cases. Hence, the mixed buffer system can be designed to provide for an optimized liposome-protein interaction.

A mixed buffer system would also provide a way of tailoring to what extent the buffer system is an interacting versus a non-interacting buffer. For example, an interacting buffer (PBS, imidazole) can be “diluted” with a non-interacting buffer (HEPES) to tailor its effect on the liposome morphology. Of course, the opposite effect (a non-interacting buffer becoming more interacting) can also be achieved by using a mixed buffer system.

Finally, in an analogous manner, one could introduce a surfactant component in the buffer composition that can assist the hydrophobic interaction of a probe with the colorimetric sensor. Surfactants that may be particularly useful in the present invention include nonionic surfactants. Polyalkoxylated, and in particular polyethoxylated, nonionic surfactants can stabilize the components of the present invention in solutions particularly well.

Surfactants of the Nonionic Type that may be useful Include:

1. Polyethylene oxide extended sorbitan monoalkylates (i.e., Polysorbates). In particular, a Polysorbate 20 commercially available as NIKKOL TL-10 (from Barret Products) is very effective.

2. Polyalkoxylated alkanols. Surfactants such as those commercially available under the trade designation BRIJ from ICI Specialty Chemicals, Wilmington, Del. having an HLB of at least about 14 have proven useful. In particular, BRIJ 78 and BRIJ 700, which are stearyl alcohol ethoxylates having 20 and 100 moles of polyethylene oxide, respectively, have proven very useful. Also useful is a ceteareth 55, which is commercially available under the trade designation PLURAFAC A-39 from BASF Corp., Performance Chemicals Div., Mt. Olive, N.J.

3. Polyalkoxylated alkylphenols. Useful surfactants of this type include polyethoxylated octyl or nonyl phenols having HLB values of at least about 14, which are commercially available under the trade designations ICONOL and TRITON, from BASF Corp., Performance Chemicals Div., Mt. Olive, N.J. and Union Carbide Corp., Danbury, Conn., respectively. Examples include TRITON X100 (an octyl phenol having 15 moles of ethylene oxide available from Union Carbide Corp., Danbury, Conn.) and ICONOL NP70 and NP40 (nonyl phenol having 40 and 70 moles of ethylene oxide units, respectively, available from BASF Corp., Performance Chemicals Div., Mt. Olive, N.J.). Sulfated and phosphated derivatives of these surfactants are also useful. Examples of such derivatives include ammonium nonoxynol-4-sulfate, which is commercially available under the trade designation RHODAPEX CO-436 from Rhodia, Dayton, N.J.

4. Polaxamers. Surfactants based on block copolymers of ethylene oxide (EO) and propylene oxide (PO) have been shown to be effective at stabilizing the film-forming polymers of the present invention and provide good wetting. Both EO-PO-EO blocks and PO-EO-PO blocks are expected to work well as long as the HLB is at least about 14, and preferably at least about 16. Such surfactants are commercially available under the trade designations PLURONIC and TETRONIC from BASF Corp., Performance Chemicals Div., Mt. Olive, N.J. It is noted that the PLURONIC surfactants from BASF have reported HLB values that are calculated differently than described above. In such situation, the HLB values reported by BASF should be used. For example, preferred PLURONIC surfactants are L-64 and F-127, which have HLBs of 15 and 22, respectively. Although the PLURONIC surfactants are quite effective at stabilizing the compositions of the present invention and are quite effective with iodine as the active agent, they may reduce the antimicrobial activity of compositions using povidone-iodine as the active agent.

5. Polyalkoxylated esters. Polyalkoxylated glycols such as ethylene glycol, propylene glycol, glycerol, and the like may be partially or completely esterified, i.e., one or more alcohols may be esterified, with a (C₈-C₂₂) alkyl carboxylic acid. Such polyethoxylated esters having an HLB of at least about 14, and preferably at least about 16, are suitable for use in compositions of the present invention.

Alkyl Polyglucosides. Alkyl polyglucosides, such as those described in U.S. Pat. No. 5,951,993 (Scholz et al.), starting at column 9, line 44, are compatible with the film-forming polymers of the present invention and may contribute to polymer stability. Examples include glucopon 425, which has a (C₈-C₁₆) alkyl chain length with an average chain length of 10.3 carbons and 1-4 glucose units.

Ultimately, the detection system based on the colorimetric materials of the present invention depends on one or more of the following factors: the molecular architecture of the diacetylene compounds; the type of receptor moiety employed; the morphology (size and structure) of the liposomes or other potential aggregate structures of diacetylene and receptor molecules; the protein probe utilized; and the buffer system used to carry out the assay.

### Methods of Detection

The present invention provides a method for analysis of an analyte, which comprises contacting the abovementioned calorimetric sensor with a solution sample or surface containing an analyte and utilizing an absorption measurement or a visual observation with the naked eye to detect color change in the calorimetric sensor.

In an alternative embodiment, the present invention provides a method for indirect detection of an analyte by selection of a probe with an affinity to bind with both the receptor incorporated into the polydiacetylene assemblies and the analyte. The probe selected will demonstrate a competitive affinity with the analyte. When the analyte of interest is present, the probe will bind to the analyte rather than the receptor on the polydiacetylene backbone, resulting in a color change inversely proportional to the analyte concentration. If the analyte is absent, the probe will bind to the receptor incorporated on the polydiacetylene backbone, resulting in a color change from blue to red. The probe can contact the sensor after the analyte contacts the sensor, or can be mixed with the analyte prior to the mixture contacting the sensor.

In an inverse detection assay, the probe and the target analyte are allowed to interact in a buffer solution, which is subsequently placed in contact with the sensor. The concentration of the probe free in the buffer is dependent on the amount of analyte target present: the higher the analyte concentration, the lower the remaining concentration of probe. Since the colorimetric response of the sensor is proportional to the amount of free probe available, the calorimetric response is inversely proportional to the analyte concentration.

In some cases, the probe can form a complex with the analyte which can interact directly with the sensor, yielding a direct assay where the calorimetric response is directly proportional to the concentration of analyte.

In one embodiment, the method of the invention comprises providing a test sample comprising the analyte in a buffer composition, providing a probe in a buffer composition, combining the test sample and the probe wherein the probe shows a greater binding affinity for the analyte than the receptor, and detecting the change with a biosensor.

It is also important to recognize that in some assays the probe could be generated in-situ by fragmenting or otherwise lysing the analyte target as discussed further below. The probe could also be considered a protein or protein fragment externally present on the cell wall of an organism that is available for interaction directly with the sensor. Interaction between the probe and the analyte can operate to the exclusion of interaction with the liposome. Alternatively the probe may interact with the analtye to form a complex with the resulting complex interacting with the liposome.

The probe can be contacted with the sensor in solution or coated on a substrate. The probe will be any molecule with an affinity for both the target analyte and the receptor. Possible probes for use in the present invention include membrane disrupting peptides such as alamethicin, magainin, gramicidin, polymyxin B sulfate, and melittin; fibrinogen; streptpyridin; antibodies; lectins; and combinations thereof.

In some embodiments, an antibody is employed as the probe. “Antibody” refers to an immunoglobulin having the capacity to specifically bind a given antigen inclusive of antigen binding fragments thereof. The term “antibody” is intended to include whole antibodies of any isotype (IgG, IgA, IgM, IgE, etc.), and fragments thereof which are also specifically reactive with a vertebrate (e.g., mammalian) protein. Antibodies can be fragmented using conventional techniques and the fragments screened for utility in the same manner as whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include F(ab′), F(ab)₂, Fv, and single chain antibodies (scFv) containing a VL and/or VH domain joined by a peptide linker. The scFv's can be covalently or non-covalently linked to form antibodies having two or more binding sites. Antibodies can be labeled with any detectable moieties known to one skilled in the art.

Various antibodies are known in the art. For example S. aureus antibodies are commercially available from Sigma and Accurate Chemical. Preferably, the concentration of antibody employed is at least 2 nanograms per milliliter (ng/ml). Typically the concentration of antibody is at least 100 nanograms/ml. For example a concentration of 100 micrograms/ml can be employed. Typically no more than about 500 micrograms/ml are employed.

In other embodiments, fibrinogen is employed as the probe. Without intending to be bound by theory, it is believed that a fibrinogen-binding protein expressed or present on/in the analyte reacts with the fibrinogen. For example, S. aureus expresses the fibrinogen binding protein often referred to as clumping factor that reacts with fibrinogen when contacted.

The concentration of fibrinogen to produce this reaction is typically at least 0.0001 wt-% and generally no more than 5 wt-%. Human plasma and animal (e.g., rabbit) plasma are suitable fibrinogen-containing mediums. Commercially available plasma products generally include an anticoagulant such as EDTA, citrate, heparin, etc. Fibrinogen derived from human is commercially available from Sigma Aldrich, St. Louis, Mo.

Using the indirect method of detection, high sensitivity that provides low levels of detection are possible based on the concentration of probe used. For detection strategy, probe concentrations can be chosen to correspond to desired concentration levels of detection. The method of indirect detection using the probe allows design of the system around the type and concentration of the probe for desired sensitivity in a given application. This allows the transducer to be universal to multiple analytes of interest. For example, a single transducer (polydiacetylene/receptor combination) could serve to detect multiple analytes by varying the probe in contact with the transducer in accordance with the probe's affinity for the analyte.

Analytes of particular interest to detect are microbes (i.e., microorganisms) such as Gram positive bacteria, Gram negative bacteria, fungi, protozoa, mycoplasma, yeast, viruses, and even lipid-enveloped viruses. Particularly relevant organisms include members of the families Enterobacteriaceae, or genera Staphylococcus spp., Streptococcus spp., Pseudomonas spp., Enterococcus spp., Esherichia spp., Bacillus spp., Listeria spp., Vibrio spp., as well as herpes virus, Aspergillus spp., Fusarium spp., and Candida spp. Particularly virulent organisms include Staphylococcus aureus (including resistant strains such as Methicillin Resistant Staphylococcus aureus (MRSA)), S. epidermidis, Streptococcus pneumoniae, S. agalactiae, S. pyogenes, Enterococcus faecalis, Vancoinycin Resistant Enterococcus (VRE), Vancomycin Resistant Staphylococcus aureus (VRSA), Vancomycin Intermediate Staphylococcus aureus (VISA), Bacillus anthracis, Pseudomonas aeruginosa, Escherichia coli, Aspergillus niger, A. fumigatus, A. clavatus, Fusarium solani, F. oxysporum, F. chlamydosporum, Listeria monocytogenes, Vibrio cholera, V. parahemolyticus, Salmonella cholerasuis, S. typhi, S. typhimurium, Candida albicans, C. glabrata, C. krusei, and multiple drug resistant Gram negative rods (MDR).

Of particular interest are Gram positive bacteria, such as Staphylococcus aureus. Typically, these can be detected by detecting the presence of a cell-wall component characteristic of the bacteria, such as a cell-wall protein. Also, of particular interest are antibiotic resistant microbes including MRSA, VRSA, VISA, VRE, and MDR. Typically, these can be detected by additionally detecting the presence of an internal cell component, such as a membrane protein.

Such microbes or other species of interest can be analyzed in a test sample that may be derived from any source, such as a physiological fluid, e.g., blood, saliva, ocular lens fluid, synovial fluid, cerebral spinal fluid, pus, sweat, exudate, urine, mucous, lactation milk, or the like. Further, the test sample may be derived from a body site, e.g., wound, skin, nares, scalp, nails, etc. As used herein “test sample” refers to a sample that contains the target analyte. Preferably, the sample is a liquid or gas and more preferably, a liquid.

The art describes various patient sampling techniques for the detection of S. aureus. Such sampling techniques are suitable for the method of the present invention as well. It is common to obtain a sample from wiping the nares of a patient. A particularly preferred sampling technique includes the subject's (e.g., patient's) anterior nares swabbed with a sterile rayon swab. One swab is used to sample each subject, i.e., one swab for both nostrils. The sampling is performed by inserting the rayon swab (commercially available from Puritan, East Grinstead, UK under the trade designation “Pure-Wraps” dry or pre-moistened with an appropriate solution into the anterior tip of the subject's nostril and rotating the swab for two complete revolutions along the nares' mucosal surface. The swab is then cultured directly or extracted with an appropriate solution typically including water optionally in combination with a buffer and at least one surfactant.

Besides physiological fluids, other test samples may include other liquids as well as solid(s) dissolved in a liquid medium. Samples of interest may include process streams, water, soil, plants or other vegetation, air, surfaces (e.g., contaminated surfaces), and the like.

The test sample (e.g., liquid) may be subjected to prior treatment, such as dilution of viscous fluids. The test sample (e.g., liquid) may be subjected to other methods of treatment prior to injection into the sample port such as concentration (by filtration, distillation, dialysis, or the like), dilution, filtration, inactivation of natural components, addition of reagents, chemical treatment, etc.

One method of treatment that may enhance signal detection of the target analyte involves lysing cells to form cell-wall fragments and analyzing the cell-wall fragments, as described in U.S. Patent Publication No. 2005/0153370. In particular, the methods are useful for detecting one or more components of cell walls that are characteristic of a microbe, particularly Staphylococcus aureus. The method includes: providing a test sample including uncultured cells; lysing the uncultured cells to form a lysate including cell-wall fragments; and analyzing the cell-wall fragments for a cell-wall component characteristic of the analyte; wherein the cell-wall component characteristic of the analyte displays an enhanced signal relative to the same component in unlysed cells.

Cell-wall components include, for example, cell-wall proteins such as protein A and microbial surface components recognizing adhesive matrix molecules (MSCRAMMs) such as fibrinogen-binding proteins (e.g., clumping factors), fibronectin-binding proteins, collagen-binding proteins, heparin/heparin-related polysaccharides binding proteins, and the like. Protein A and clumping factors, such as fibrinogen-binding factors and clumping factors A, B, and Efb, are also particularly useful in methods of detecting the presence of Staphylococcus aureus. Other cell-wall components include capsular polysaccharides and cell-wall carbohydrates (e.g., teichoic acid and lipoteichoic acid).

Lysing can include contacting the cells with a lysing agent or physically lysing the cells. Lysing can be conducted under conventional conditions, such as, for example, at a temperature of about 5° C. to about 37° C., preferably at a temperature of about 15° C. to about 25° C. Significantly, the lysing can occur using uncultured cells, i.e., a direct test sample, although cultured cells can be used as well.

As a result of lysing the cells to form cell-wall fragments and the resultant enhancement of the signal of cell-wall components, samples having relatively low concentrations of the species of interest can be evaluated. For example, for certain embodiments, the test sample may include a relatively low concentration of microbes, particularly Staphylococcus aureus. Such relatively low concentrations include, for example, less than about 5×10⁴ colony forming units (“cfu”) per milliliter (cfu/ml) of microbe, less than about 5×10³ cfu/ml, less than about 1000 cfu/ml, and even as low as about 500 cfu/ml. Microbes, such as S. aureus, can be detected at high levels as well, ranging up to as much as 5×10⁷ cfu/ml, for example.

Suitable lysing agents include, for example, enzymes such as lysostaphin, lysozyme, endopeptidases, N-acetylmuramyl-L-alanine amidase, endo-beta-N-acethylglucosaminidase, and ALE-1. Various combinations of enzymes can be used if desired. Lysostaphin is particularly useful in methods of detecting the presence of Staphylococcus aureus.

Other lysing agents include salts (e.g., chaotrophic salts), solubilizing agents (e.g., detergents), reducing agents (e.g., DTT, DTE, cysteine, N-acetyl cysteine), acids (e.g., HCLI), bases (e.g., NaOH). Various combinations of such lysing agents can be used if desired.

One example is if S. aureus is present, the lysed cells in the test sample can be analyzed for protein A, which is characteristic for S. aureus and can be detected with a protein A specific antibody immobilized on the biosensor surface. Additionally, lysed cells, such as S. aureus bacteria, release protein markers from the internal portion of the cells (as opposed to the cell-wall portion of the cells). Such protein markers can be detected by probes, such as an antibody.

The test sample and probe may be combined in a variety of suitable manners. In one aspect, the probe is provided to the sensor and the test sample is provided to the calorimetric sensor as separate portions, yet in any order. For example the surface may be coated with a fibrinogen-containing solution and optionally dried. In another aspect, the test sample and probe are combined as a mixture and the mixture is provided to the calorimetric sensor. In a preferred embodiment, the probe interacts with the test sample containing the analyte before contacting the calorimetric sensor.

Advantageously, the method of the invention has improved sensitivity. As further described in the forthcoming examples, S. aureus can be detected at concentrations of 5×10⁴ colony forming units (“cfu”) per milliliter, 5×10³ cfu/ml, and 5×10² cfa/ml. Accordingly, one of ordinary skill in the art appreciates that the method of the present invention can be employed to detect a target analyte at concentrations as low as 5×10² cfu/ml (e.g., any specific concentration between the stated concentrations at increments of 10 cfu/ml). A target analyte can also be detected at high levels as well, ranging up to as much as 5×10⁷ cfu/ml.

Alternatively, or in addition thereto, the method of the invention also advantageously result in an improved detection rate. The device employed herein is capable of detecting an analyte in a relatively short period of time. For example, S. aureus can be detected at any of the concentrations previously described in less than 120 minutes (e.g. 90 minutes, 60 minutes, 30 minutes, 10 minutes).

### Applications

The calorimetric sensors of the present invention formed from the disclosed diacetylene compounds are amenable to a variety of applications that demand cost-effective, stable, accurate, consistent and quick diagnostics outside the laboratory setting. Applications include point-of-care testing, home testing diagnostics, military and industrial detection of air- or water-borne pathogens and VOCs, and food processing.

In one embodiment, the calorimetric sensors can be used for the detection of gram-negative bacteria in biological fluids to diagnose the presence of an infection. For example, the presence of gram-negative bacteria in urine is indicative of a urine infection. A calorimetric sensor comprising the polydiacetylene assemblies of the present invention can indicate the presence of gram-negative bacteria such as S. aureus in biological fluids through color change either in a solution or as a coating on a substrate.

In certain embodiments, the colorimetric sensors of the present invention could be paired with other known diagnostic methods to provide a multi-prong determination of the presence of bacteria or other analytes.

In one embodiment, the calorimetric sensors of the present invention could be used in conjunction with wound dressings to detect the presence of an infection. The sensor could be integrated in the dressing as a layer directly or indirectly in contact with the wound. The sensor could also be inserted into the dressing during use. Alternatively, one could conceive a dressing construction where wound exudate could be directed from the wound to a portion of the dressing not contacting the wound where the sensor is located, through microfluidic channels such as those described in U.S. Pat. No. 6,420,622 B1. The sensor could also be used as a stand-alone diagnostic in the assessment of a wound infection by analyzing the analyte extracted from a wound swab.

A sensor comprising the polydiacetylene assemblies can be obtained without the need to form a film by the conventional LB (Langmuir-Blodgett) process before transferring it onto an appropriate support. Alternatively, the polydiacetylene assemblies can be formed on a substrate using the known LB process as described in A. Ulman, An Introduction to Ultrathin Organic Films, Academic Press, New York (1991), pp. 101-219.

The present invention can provide biosensing capabilities in a disposable adhesive product. The sensors are self-contained and do not require additional instrumentation to convey a measurable result. Alternatively, use with other analytical instrumentation is possible to further enhance sensitivity, such as fluorescence with the fluorescent “red” phase developed after detection of the analyte. The sensors function to provide a rapid screening device, i.e., less than 30 minutes, and preferably less than 15 minutes, when the detection of a threshold presence of a specific analyte is desired. Additionally, the sensors of the present invention are disposable and relatively inexpensive.

In one embodiment of the invention, the colorimetric sensor comprises a transducer formed from a receptor incorporated within the polydiacetylene assemblies in solution. The solution can be provided in a simple vial system, with the analyte directly added to a vial containing a solution with the transducer specific to the analyte of interest. Alternatively, the calorimetric sensor could comprise multiple vials in a kit, with each vial containing a transducer comprising polydiacetylenes assemblies with incorporated receptors particular to different analytes. For those applications in which the analyte cannot be added directly to the polydiacetylene transducer, a two-part vial system could be used. One compartment of the vial could contain reagents for sample preparation of the analyte physically separated from the second compartment containing the transducer formed from the polydiacetylene assemblies. Once sample preparation is complete, the physical barrier separating the compartments would be removed to allow the analyte to mix with the transducer for detection.

The calorimetric sensor as prepared can then be coated on a solid substrate by either spotting the substrate and allowing water to evaporate, or extruding the suspension through a membrane of appropriate pore size, entrapping the polydiacetylene assemblies and resulting in a coated membrane, which is subsequently allowed to dry. Appropriate membranes are generally those with pore size of 200 nm or less, comprising materials like polycarbonate, nylon, PTFE, polyethylene (others can be listed). These substrates can be either coated with a polymerized suspension of the diacetylene assemblies, or the suspension can be coated in the un-polymerized form and subsequently polymerized in the coated state.

In another embodiment of the present invention, the calorimetric sensor is a rapid indicator in a tape or label format as depicted in FIG. 1. FIG. 1 shows a tape or label 10 coated with a pressure sensitive adhesive 20 and a transducer 30 coated on a substrate 40. Suitable substrates for use with the present invention can be chracteractered by contact angle measurements using millli-Q (Millipore) water and methylene iodide (Aldrich) as described in U.S. Published Application No. 2004-0132217-A1.

Substrate 40 can include highly flat substrates, such as evaporated gold on atomically flat silicon (111) wafers, atomically flat silicon (111) wafers, or float glass, which are bare and modified with self-assembling monolayers (SAMs) to alter their surface energy in a systematic fashion; or substrates with a highly textured topography that include paper substrates, polymeric ink receptive coatings, structured polymeric films, microporous films, and membrane materials.

In an embodiment of the invention that maintains the original “blue” phase of the polydiacetylene assemblies upon drying, the substrate 40 exhibits advancing contact angles with methylene iodide below 50°. This condition corresponds to substrates characterized by a dispersive component of their surface energy greater than 40 dynes/cm. In an alternate embodiment, substrates with these properties that have an advancing contact angle with water less than 90° result in dry coatings containing a mixture of the blue and red phases. This condition would correspond to surfaces in which the dispersive surface energy component could be less than 40 dynes/cm but with a polar surface energy component greater than at least 10 dynes/cm.

Referring again to FIG. 1, pressure sensitive adhesive 20 can affix tape or label 10 to a surface for direct detection of an analyte. Pressure sensitive adhesive 20 is isolated from transducer 30 containing the polydiacetylene assemblies to potentially minimize adverse effects. In FIG. 1, pressure sensitive adhesive 20 surrounds the transducer 30 located in the center of tape or label 10. In an alternate embodiment (not shown), the pressure sensitive adhesive and the transducer are combined.

Optionally, tape or label 10 will contain a transparent window on the side of tape or label 10 that does not contain pressure sensitive adhesive 20. The window would be centered under transducer 30 to allow the user to view the color change without removing the tape or label 10 from the surface containing the analyte.

In FIG. 2, the tape or label 110 is shown as array 111 composed of multiple transducers 112, 113, 114, 115, and 116. Each of transducers 112, 113, 114, 115, and 116 could be formed from the same or different polydiacetylene assemblies with each polydiacetylene assembly incorporating the same or different receptor. By varying transducers 112, 113, 114, 115, and 116, array 111 can be designed to detect multiple analytes at various concentration levels. Alternatively, any one of transducers 112, 113, 114, 115 can be replaced with an alternative diagnostic test. Other embodiments contemplated with the present invention are provided in U.S. Ser. No. 10/738,573.

For those applications requiring sample preparation of the analyte, a kit could contain a vial for reagant storage and mixing of the analyte before contacting the calorimetric sensor coated on a two-dimensional substrate. In one embodiment, the kit could comprise a vial for reagent storage and analyte preparation, with a cap system containing the transducer of the present invention coated on a substrate.

### EXAMPLES

The present invention should not be considered limited to the particular examples described below, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the instant specification. All parts, percentages, ratios, etc. in the examples and the rest of the specification are by mole unless indicated otherwise. All solvents and reagents without a named supplier were purchased from Aldrich Chemical; Milwaukee, Wis. Water was purified by the use of a U-V Milli-Q water purifier with a resistivity of 18.2 Mohms/cm. (Millipore, Bedford Mass.)

Colorimetric response (CR) was determined using a picture taken using a digital camera. The picture was scanned using software from Adobe Systems Incorporated (trade designation ADOBE PHOTOSHOP version 5.0, San Jose, Calif.) to obtain the RGB (Red, Green, Blue) channel values for each polydiacetylene sensor test. The red and blue channel values as given by the equation CR=((PRinitial−PRsample)/PRinitial) where PR=percent red value of the sample, and is given by the equation PR=Rᵥₐₗᵤₑ(Rᵥₐₗᵤₑ+Bᵥₐₗᵤₑ)*100, where Rᵥₐₗᵤₑ and Bᵥₐₗᵤₑ correspond to the value of the polydiacetylene sensor's red and blue channel respectively.

Table of Abbreviations

| Abbreviation or Trade Name | Description |
|---|---|
| ATCC | American Type Culture Collection |
| DMPC | 1,2-dimeristoyl-sn-glycero-3- |
| | phosphocholine (DMPC, formula weight |
| | (F.W.) 678, available from Sigma- |
| | Aldrich, St. Louis, MO |
| HEPES | N-2-Hydroxyethylpiperazine-N′-2- |
| | ethanesulfonic acid available from Sigma- |
| | Aldrich, St. Louis, MO |
| Imidazole buffer solution | 30 mM Imidazole, 125 mM Sodium |
| | Chloride, 0.1% (w/v) Sodium Azide in |
| | water, pH = 7.3, available commercially |
| | from Sigma Diagnostics, cat. No I2900 |
| PBS buffer | A phosphate buffer saline (PBS) solution |
| | prepared by diluting ten-fold a 10x PBS |
| | liquid concentrate available commercially |
| | from EMD Biosciences, San Diego CA |
| PBS L64 buffer | prepared by taking the PBS buffer |
| | solution and adding 0.2% (w/v) of |
| | PLURONIC L64 |
| PLURONIC L64 | Trade designation for surfactant available |
| | from BASF Corporation, Mount Olive, NJ |
| Tricosadiynoic acid | Available from GFS Chemicals (Powell, |
| | OH) |

### Preparative Example 1

### Preparation of a Suspension of Diacetylene Liposomes

Diacetylene, HO(O)C(CH₂)₂C(O)O(CH₂)₄C≡C—C≡C(CH₂)₄O(O)C(CH₂)₁₂CH₃ was prepared as in Example 6 of U.S. Patent Application Publication No. 2004/0132217. The basic procedure involved reacting 5,7-dodecadiyn-1,12-diol (HO(CH₂)₄C≡C—C≡C(CH₂)₄OH) with myristol chloride and subsequent reaction of that product with succinic anhydride to yield the diacetylene, HO(O)C(CH₂)₂C(O)O(CH₂)₄C≡C—CC(CH₂)₄O(O)C(CH₂)₁₂CH₃ as a white solid.

A (6:4) mixture of the diacetylene compound: HO(O)C(CH₂)₂C(O)O(CH₂)₄C≡C—C≡(CH₂)₄O(O)C(CH₂)₁₂CH₃ (succinic acid mono-(12-tetradecanoyloxy-dodeca-5,7-diynyl) ester), and the zwitterionic phospholipid 1,2-dimeristoyl-sn-glycero-3-phosphocholine (DMPC, formula weight (F.W.) 678, available from Sigma-Aldrich, St. Louis, Mo.) was weighed into a glass vial and suspended in N-2-Hydroxyethylpiperazine-N′-2-ethanesulfonic acid (HEPES) buffer (5 mM, pH 7.2) to produce a 1 mM solution. This solution was then probe sonicated using a Misonix XL202 probe sonicator (available commercially from Misonix Inc., Farmington, N.Y.) for 2 minutes, and placed in a 4° C. refrigerator for about 20 hours. This process results in the formation of a stable liposome suspension.

### Preparative Example 2

### Polymerization of the Diacetylene Liposome Suspension

The suspension prepared in Preparative Example 1 was filtered through a 1.2 μm syringe filter and polymerized by irradiating the sample beneath a 254 nm UV lamp (commercially available from VWR Scientific Products; West Chester, Pa.) at a distance of 3 cm for 10 minutes, resulting in the observation of a blue color being developed.

### Preparative Example 3

### Preparation of Coated Samples of the Diacetylene Liposome Suspension

The suspension prepared in Preparative Example 1 was coated onto 25 (mm) diameter porous polycarbonate membranes with 200 (nm) diameter pores (Avestin, Inc. Ottawa, Canada) to make colorimetric detector samples. The membranes were coated using a handheld extrusion process as follows. The polycarbonate membrane to be coated was placed into the stainless steel chamber of a handheld extruder system, trade designation LIPOFAST, available from Avestin, Inc. (Ottawa, Canada). The membrane covered the bottom O-ring of the TEFLON base. Care was taken to avoid bending and/or creasing the membrane. The top TEFLON O-ring block was placed inside the stainless steel housing on top of the membrane. The chamber was then sealed by tightening the stainless steel caps by hand. A Gas Tight syringe (Hamilton 500-microliter (μl)) was filled with a suspension of diacetylene liposomes and attached to the base and a second syringe was attached to the other cap. The liposomes of the first syringe were forced slowly through the chamber with constant even pressure.

The membrane captured the liposomes on the surface allowing the clear buffer to flow slowly through and into second syringe. This action was considered a 1 pass coating. The membrane samples used as detectors in this example used 2 passes of coating. The second pass was applied like the first by a second 0.5 milliliter (ml) portion of liposome being applied to the already coated membrane. The second syringe containing the filtered buffer was removed and the contents were discarded. The stainless steel end cap was unscrewed and the TEFLON O-ring block removed. The wet membrane was removed and placed coated side up on a glass slide and placed in a refrigerator at 5° C. for at least 3 hours. The sample was then dried in a dessiccator containing CaSO₄ for 30 minutes and exposed to 254 nanometer (nm) UV light for 30-90 seconds.

The PDA-coated substrate (25 millimeter (mm) circle) was cut into four quarters. Each quarter sample was used as a sample for an experiment.

### Preparative Example 4

### Preparation of Phospate Buffer Saline (PBS buffer solution)

A phosphate buffer saline (PBS) solution was prepared by diluting ten-fold a 10× PBS liquid concentrate (available commercially from EMD Biosciences, San Diego Calif.). This results in a PBS buffer solution with the following salt composition: 10 mM Sodium Phosphate, 137 mM Sodium Chloride, 2.7 mM Potassium Chloride. The PBS buffer solution has a pH of 7.5 at 25° C.

### Preparative Example 5

### Preparation of Phospate Buffer Saline with PLURONIC L64 (PBS L64 Buffer Solution)

PBS L64 buffer solution was prepared by taking the PBS buffer solution as prepared in Preparative Example 4 and adding 0.2% (w/v) of PLURONIC L64 surfactant (available from BASF Corporation, Mount Olive, N.J.). The PBS L64 buffer solution has a pH of 7.5 at 25° C.

### Preparative Example 6

### S. aureus Bacteria Suspension Preparation

S. aureus bacteria were obtained from The American Type Culture Collection (Rockville, Md.), under the trade designation “ATCC 25923.” The bacteria were grown in overnight (17-22 hours at 37° C.) broth cultures prepared by inoculating 5-10 milliliters of prepared, sterile Tryptic Soy Broth (Hardy Diagnostics, Santa Maria, Calif.) with the bacteria. Cultures were washed by centrifugation (8,000-10,000 rpm for 15 minutes in an Eppendorf model number 5804R centrifuge (Brinkman Instruments, Westbury, N.Y.) and resuspended into PBS L64 buffer and washed by centrifugation for 3 additional cycles with this solution.

### Preparative Example 7

### S. epidermidis Bacteria Suspension Preparation

S. epidermidis bacteria were obtained from The American Type Culture Collection (Rockville, Md.), under the trade designation “ATCC 12228.” The bacteria were grown in overnight (17-22 hours at 37° C.) broth cultures prepared by inoculating 5-10 milliliters of prepared, sterile Tryptic Soy Broth (Hardy Diagnostics, Santa Maria, Calif.) with the bacteria. Cultures were washed by centrifugation (8,000-10,000 rpm for 15 minutes in an Eppendorf model number 5804R centrifuge (Brinkman Instruments, Westbury, N.Y.) and resuspended into PBS L buffer and washed by centrifugation for 3 additional cycles with this solution.

### Preparative Example 8

### E. coli Bacteria Suspension Preparation

E. coli bacteria were obtained from The American Type Culture Collection (Rockville, Md.), under the trade designation “ATCC 25922.” The bacteria were grown in overnight (17-22 hours at 37° C.) broth cultures prepared by inoculating 5-10 milliliters of prepared, sterile Tryptic Soy Broth (Hardy Diagnostics, Santa Maria, Calif.) with the bacteria. Cultures were washed by centrifugation (8,000-10,000 rpm for 15 minutes in an Eppendorf model number 5804R centrifuge (Brinkman Instruments, Westbury, N.Y.) and resuspended into HEPES buffer and washed by centrifugation for 3 additional cycles with this solution.

### Example 1

### Solution Phase Detection of Fibrinogen Protein Probe

Fibrinogen from human plasma (available from Sigma Aldrich, St. Louis, Mo., cat. No FR4129) was dissolved in Imidazole buffer at a concentration of 0.5% (w/v). Fibrinogen in imidazole buffer solution (100 μl) was mixed with 100 μl of the blue polydiacetylene liposome solution as prepared in Preparative Example 2. A control sample containing 100 μl of imidazole buffer solution without fibrinogen and 100 μl of the blue polydiacetylene liposome solution as prepared in Preparative Example 2 was also prepared. Although both samples changed from blue to red in the first 20 minutes, the suspension sample containing fibrinogen went on to flocculate and subsequently precipitate in a total of 30 minutes. The suspension of the control sample remained stable over the entire observation time.

### Example 2

### Solution phase detection of Rabbit anti Staphylococcus aureus IgG Antibody Protein Probe

Rabbit anti Staphylococcus aureus IgG antibody (obtained from Accurate Chemical and Scientific Corporation, Westbury, N.Y., cat.No. YVS6881) was dissolved in Imidazole buffer solution at a concentration of 100 μg/ml. The antibody in imidazole buffer solution (100 μl) was mixed with 100 μl of the blue polydiacetylene liposome solution (prepared in Preparative Example 2). A control sample containing 100 μl of imidazole buffer solution without antibody and 100 μl of the blue polydiacetylene liposome solution (prepared in Preparative Example 2) was also prepared. Although both samples changed from blue to red in the first 30 minutes, the suspension sample containing the antibody went on to flocculate and subsequently precipitate after 24 hours. The suspension of the control sample remained stable over the entire observation time.

### Example 3

### Solution Phase Detection of the Fibrinogen Protein Probe in the Presence of S. aureus and PBS L64 Buffer Solution

Fibrinogen in imidazole buffer solution (100 μl) as prepared in Example 1, was mixed with 100 μl of the blue polydiacetylene liposome solution (prepared as in Preparative Example 2) and 100 μl of PBS L64 buffer solution containing 10⁶ cfu/ml of S. aureus bacteria as prepared in Preparative Example 6. A control sample was also prepared by mixing 100 μl of fibrinogen in imidazole buffer solution, 100 μl of the blue polydiacetylene liposome solution, and 100 μl of PBS L64 buffer solution without S. aureus bacteria. Both samples changed from blue to red in 30 minutes, but in contrast to Example 1, the suspensions remained stable for both samples over a 24-hour observation period.

### Example 4

### Solution Phase Detection of the Rabbit Anti Staphylococcus aureus IgG Antibody Protein Probe in the Presence of S. aureus and PBS L64 Buffer Solution

The blue polydiacetylene liposome solution as prepared in Preparative Example 2 was mixed with the antibody in imidazole buffer solution as prepared in Example 2 and PBS buffer solution containing S. aureus bacteria as prepared in Preparative

### Example 6, using three different combinations

Sample 4A—100 μl of blue polydiacetylene liposome solution+100 μl antibody in imidazole buffer solution+100 μl of PBS buffer solution containing 107 cfa/ml S. aureus bacteria.

Sample 4B—100 μl of blue polydiacetylene liposome solution+100 μl antibody in imidazole buffer solution+100 μl of PBS buffer solution without bacteria.

Sample 4C—100 μl of blue polydiacetylene liposome solution+100 μl imidazole buffer solution without antibody+100 μl of PBS buffer solution without bacteria.

The sample's color after 45 minutes is recorded in Table 1 below.

TABLE 1

| Sample | Color @ 45 minutes |
|---|---|
| 4A | Purple |
| 4B | Light Red |
| 4C | Red |

### Example 5

### Detection of Fibrinogen Protein Probe using Coated Samples of Polydiacetylene

Three polydiacetylene coated substrates as prepared in Preparative Example 3 were placed in the bottom of a well in a 24-well microtiter plate (available commercially from Corning Incorporated, Corning N.Y., cat. No 3524 under the trade designation COSTAR), and the following solutions were added:

Sample 5A—250 μl of fibrinogen in imidazole buffer solution as prepared in Example 1+250 μl of PBS L64 buffer solution.

Sample 5B—250 μl of fibrinogen in imidazole buffer solution+250 μl of PBS L64 buffer solution containing 10⁷ cfa/ml of S. aureus bacteria as prepared in Preparative Example 6.

Sample 5C—250 μl of fibrinogen in imidazole buffer solution+250 μl of PBS L64 buffer solution containing 10⁷ cfu/ml of S. epidermidis as prepared in Preparative Example 7.

The time required for each sample to change from blue to red is recorded in Table 2 below.

TABLE 2

| Sample | Time to red (in minutes) |
|---|---|
| 5A | 2 |
| 5B | 15 |
| 5C | 5 |

### Example 6

### Detection of S. aureus in PBS L64 Buffer Solution at Various Concentrations using a Fibrinogen Protein Probe in Imidazole Buffer Solution

Six polydiacetylene coated substrates as prepared in Preparative Example 3 were placed at the bottom of separate wells in a 24-well microtiter plate. Fibrinogen in imidazole buffer solution (250 μl) as prepared in Example 1 was mixed with 250 μl of PBS L64 buffer solution containing S. aureus bacteria as prepared in Preparative Example 6, yielding a series of sample mixtures containing various concentrations of bacteria. The bacteria concentration is listed in Table 3 below. The different sample mixtures were vortexed and allowed to stand for 5 minutes and then added to separate wells containing the polydiacetylene coated substrates. The microtiter plate was agitated on an Eberbach Model 6000 shaker (Eberbach Corp., Ann Arbor, Mich.). A picture was taken at 6 minutes using a digital camera. The picture was scanned using software from Adobe Systems Incorporated. Colorimetric response (CR) was determined as described above. The data in Table 3 below reports the calorimetric response as a function of the bacteria concentration.

TABLE 3

| | S. aureus Concentration | Colorimetric Response |
|---|---|---|
| Sample | (cfu/ml) | (Fraction Red) |
| 6A | 0 | 2.4 |
| 6B | 100 | 2.4 |
| 6C | 1000 | 2.4 |
| 6D | 10000 | 1.8 |
| 6E | 100000 | 1.6 |
| 6F | 1000000 | 1.4 |

### Example 7

### Detection of S. aureus in PBS L64 Buffer Solution using an Antibody-Streptavidin Conjugated Protein Probe and Coated Samples of Polydiacetylene

Two polydiacetylene-coated substrates as prepared in Preparative Example 3 were placed at the bottom of separate wells of a 24-well microtiter plate. A streptavidin conjugated Rabbit anti Staphylococcus aureus IgG antibody protein probe was prepared in the following manner. The streptavidin conjugated antibody was dissolved in imidazole buffer solution at a concentration of 100 μg/ml.

The Following Sample Solutions were then Prepared:

Sample 7A—250 μl of streptavidin conjugated antibody in imidazole buffer solution+250 μl of PBS buffer solution as prepared in Preparative Example 4.

Sample 7B—250 μl of streptavidin conjugated antibody in imidazole buffer solution+250 μl of PBS buffer solution containing 10⁶ cfu/ml S. aureus bacteria in PBS buffer solution as prepared in Preparative Example 6.

The solutions were vortexed and allowed to stand 5 minutes after mixing, and then were added to separate wells containing the polydiacetylene sensors. The microtiter plate was agitated on an Eberbach Model 6000 shaker (Eberbach Corp., Ann Arbor, Mich.). Table 4 below records the time required for each sensor to change from blue to red.

TABLE 4

| | Time to red |
|---|---|
| Sample | (in minutes) |
| 7A | 9 |
| 7B | 20 |

### Example 8

### Detection of Streptavidin using an Antibody-Biotin Conjugated Protein Probe using Coated Samples of Polydiacetylene

Four polydiacetylene-coated substrates as prepared in Preparative Example 3 were placed at the bottom of separate wells of a 24-well microtiter plate. A biotin conjugated mouse anti protein A IgG monoclonal antibody (available commercially from Sigma Aldrich, St. Louis, Mo., cat. No 13-3150) protein probe was dissolved in PBS buffer solution at a concentration of 100 μg/ml. Streptavidin (available commercially from Jackson Immuno Research, West Grove, Pa., Cat. No 016-050-084) was dissolved in PBS buffer solution at a concentration of 100 μg/ml.

The Following Sample Solutions were then Prepared:

Sample 8A—300 μl of imidazole buffer solution.

Sample 8B—150 μl of imidazole buffer solution+150 μl of streptavidin in PBS buffer solution.

Sample 8C—100 μl of imidazole buffer solution+100 μl of streptavidin in PBS buffer solution+100 μl of biotin conjugated antibody in PBS buffer solution.

Sample 8D—150 μl of imidazole buffer solution+150 μl of biotin conjugated antibody in PBS buffer solution.

The solutions were vortexed and allowed to stand 5 minutes after mixing, and then were added to separate wells containing the polydiacetylene sensors. The microtiter plate was agitated on an Eberbach Model 6000 shaker (Eberbach Corp., Ann Arbor, Mich.). Table 5 below records the time required for each sensor to change from blue to red.

TABLE 5

| | Time to red |
|---|---|
| Sample | (in minutes) |
| 8A | 13 |
| 8B | 9 |
| 8C | 6 |
| 8D | 13 |

### Example 9

### Detection of S. aureus in PBS L64 Buffer Solution at Various Concentrations using a Fibrinogen Protein Probe in PBS L64 Buffer Solution

Six polydiacetylene-coated substrates as prepared in Preparative Example 3 were placed at the bottom of separate wells in a 24-well microtiter plate. Fibrinogen was dissolved in PBS L64 buffer solution, at a concentration of 0.5% (w/v). Similarly, fibrinogen was also dissolved in PBS L64 buffer solution at a concentration of 0.05% (w/v).

The Following Sample Solutions were Prepared:

Sample 9A—250 μl of fibrinogen in PBS L64 buffer solution at a concentration of 0.5%+250 μl of PBS L64 buffer solution without bacteria.

Sample 9B—250 μl of fibrinogen in PBS L64 buffer solution at a concentration of 0.5%+250 μl of PBS L64 buffer solution containing 103 cfu/ml S. aureus bacteria as prepared in Preparative Example 6.

Sample 9C—250 μl of fibrinogen in PBS L64 buffer solution at a concentration of 0.5%+250 μl of PBS L64 buffer solution containing 105 cfu/ml S. aureus bacteria.

Sample 9D—250 μl of fibrinogen in PBS L64 buffer solution at a concentration of 0.05%+250 μl of PBS L64 buffer solution without bacteria.

Sample 9E—250 μl of fibrinogen in PBS L64 buffer solution at a concentration of 0.05%+250 μl of PBS L64 buffer solution containing 10³ cfu/ml S. aureus bacteria.

Sample 15F—250 μl of fibrinogen in PBS L64 buffer solution at a concentration of 0.05%+250 μl of PBS L64 buffer solution containing 10⁵ cfu/ml S. aureus bacteria.

For comparative purposes two other samples were also prepared:

Sample 9G—250 μl of fibrinogen in PBS L64 buffer solution at a concentration of 0.5%+250 μl of PBS L64 buffer solution containing 10⁵ cfu/ml S epidermidis bacteria as prepared in Preparative Example 7.

Sample 9H—250 μl of fibrinogen in PBS L64 buffer solution at a concentration of 0.05%+250 μl of PBS L64 buffer solution containing 10⁵ cfu/ml S. epidermidis bacteria.

The different sample mixtures were vortexed and allowed to stand for 5 minutes and then added to separate wells containing the polydiacetylene coated substrates. The microtiter plate was agitated on an Eberbach Model 6000 shaker (Eberbach Corp., Ann Arbor, Mich.). A picture was taken at 30 minutes using a digital camera. The picture was scanned using software from Adobe Systems Incorporated (trade designation ADOBE PHOTOSHOP version 5.0, San Jose, Calif.). The data in Table 6 below reports the calorimetric response (CR) for these samples.

TABLE 6

| | Fibrinogen | | | |
|---|---|---|---|---|
| | Concentration | | | Colorimetric |
| | (% w/v in PBS | | Bacteria | Response |
| | L64 buffer | | Concentration | (Fraction |
| Sample | solution) | Bacteria Type | (cfu/ml) | Red) |
| 9A | 0.5 | None | 0 | 2.3 |
| 9B | 0.5 | S. aureus | 1000 | 2.1 |
| 9C | 0.5 | S. aureus | 100000 | 0.9 |
| 9D | 0.05 | None | 0 | 3.3 |
| 9E | 0.05 | S. aureus | 1000 | 2.7 |
| 9F | 0.05 | S. aureus | 100000 | 1.6 |
| 9G | 0.5 | S. epidermidis | 100000 | 3.1 |
| 9H | 0.05 | S. epidermidis | 100000 | 3.2 |

### Example 10

### Detection of Whole S. aureus in Clinical Samples using a Fibrinogen Protein Probe in PBS L64 Buffer Solution

Nasal swab samples from 6 patients were collected, two swabs collected from each patient for a total of 12 samples. The nasal swab samples were obtained by wiping the anterior nares of a patient using a sterile rayon swab (commercially available from Puritan, East Grinstead, UK under the trade designation “Pure-Wraps”). Sampling was performed by inserting the rayon swab into the anterior tip of the subject's nares and rotating the swab for two complete revolutions along the nares' mucosal surface. Each swab sample was eluted using 1 ml of PBS L64 buffer solution. One sample each from 6 patients was analyzed using the coated polydiacetylene sensors as prepared in Preparative Example 3. The second sample from the same patient was eluted using 1 ml PBS L64 buffer solution and cultured to obtain a bacterial count for comparison that is reported in Table 7 below. The culture procedure for these examples follows that is generally described in The Staphylococci in Human Disease; Crossley, K. B. and Archer, G. L. editors, Churchill Livingston, N.Y., 1997, pp. 233-252. The samples to be analyzed with the polydiacetylene sensors were prepared by mixing 250 μl of fibrinogen dissolved in PBS L64 buffer solution at a concentration of 0.5% (w/v) and 250 μl of the solution eluted from each patient swab. The sample solution was vortexed and allowed to stand for 5 minutes and then placed over the polydiacetylene coated sensors, which had been placed at the bottom of separate wells in a 24-well microtiter plate. The microtiter plate was agitated on an Eberbach Model 6000 shaker (Eberbach Corp., Ann Arbor, Mich.). A picture was taken at 45 minutes using a digital camera. The picture was scanned using software from Adobe Systems Incorporated (trade designation ADOBE PHOTOSHOP version 5.0, San Jose, Calif.). The data in Table 7 below reports the calorimetric response as a function of the bacteria concentration.

TABLE 7

| | Culture Bacterial Count | Colorimetric Response |
|---|---|---|
| Swab Sample | (cfu) | (Fraction Red) |
| 10A | 0 | 1.3 |
| 10B | 25 | 1.2 |
| 10C | 631 | 0.9 |
| 10D | 1995 | 0.9 |
| 10E | 39811 | 0.7 |
| 10F | 125892 | 0.7 |

### Example 11

### Detection of Lysed S. aureus in Clinical Samples using a Fibrinogen Protein Probe in PBS L64 Buffer Solution

Nasal swab samples from 5 patients were collected, two swabs collected from each patient for a total of 10 samples. The samples were obtained as in Example 10. One sample each from 5 patients was analyzed using the coated polydiacetylene sensors as prepared in Preparative Example 3. The second sample from the same patient was eluted using 1 ml PBS L64 buffer solution and cultured to obtain a bacterial count as described in Example 10. The samples to be analyzed with the polydiacetylene sensors were prepared as follows. First, the S. aureus bacteria present in the 1 ml eluted swab sample was lysed by mixing with an equivalent volume of a lysing buffer solution consisting of lysostaphin (catalog number L-4402, Sigma-Aldrich) in PBS L64 buffer solution at a concentration of 3 μg/ml. Second, 250 μl of the lysed solution was mixed with 250 μl of fibrinogen dissolved in PBS L64 buffer solution at a concentration of 0.5% (w/v). The sample solution was vortexed and allowed to stand for 5 minutes and then placed over the polydiacetylene coated sensors that had been placed at the bottom of separate wells in a 24-well microtiter plate. The microtiter plate was agitated on an Eberbach Model 6000 shaker (Eberbach Corp., Ann Arbor, Mich.). A picture was taken at 42 minutes using a digital camera. The picture was scanned using software from Adobe Systems Incorporated (trade designation ADOBE PHOTOSHOP version 5.0, San Jose, Calif.). The data in Table 8 below reports the calorimetric response as a function of the bacteria concentration.

TABLE 8

| | Culture Bacterial Count | Colorimetric Response |
|---|---|---|
| Swab Sample | (cfu) | (Fraction Red) |
| 11A | 0 | 1.0 |
| 11B | 63 | 0.9 |
| 11C | 160 | 1.2 |
| 11D | 7940 | 1.4 |
| 11E | 40000 | 2.0 |

### Example 12

### Detection of Lysed S. aureus in Clinical Samples using a Rabbit Anti Staphylococcus aureus IgG Antibody Protein Probe in PBS L64 Buffer Solution

Nasal swab samples from 6 patients were collected, two swabs collected from each patient for a total of 12 samples. Sampling was done as described in Example 10 One sample each from 6 patients was analyzed using the coated polydiacetylene sensors as prepared in Preparative Example 3. The second sample from the same patient was eluted using 1 ml PBS L64 buffer solution and cultured to obtain a bacterial count for comparison that is reported in Table 9 below. The culture procedure was done as described in Example 10. The samples to be analyzed with the polydiacetylene sensors were prepared as follows. First, the S. aureus bacteria present in the 1 ml eluted swab sample was lysed by mixing with an equivalent volume of a lysing buffer solution consisting of lysostaphin (catalog number L-4402, Sigma-Aldrich) in PBS L64 buffer solution at a concentration of 3 μg/ml. Second, 250 μl of the lysed solution was mixed with 250 μl of Rabbit anti Staphylococcus aureus IgG antibody (obtained from Accurate Chemicals) dissolved in PBS L64 buffer solution at a concentration of 100 μg/ml. The sample solution was vortexed and allowed to stand for 5 minutes and then placed over the polydiacetylene coated sensors, which had been placed at the bottom of separate wells in a 24-well microtiter plate. The microtiter plate was agitated on an Eberbach Model 6000 shaker (Eberbach Corp., Ann Arbor, Mich.). A picture was taken at 20 minutes using a digital camera. The picture was scanned using software from Adobe Systems Incorporated (trade designation ADOBE PHOTOSHOP version 5.0, San Jose, Calif.). The data in Table 9 below reports the colorimetric response as a function of the bacteria concentration.

TABLE 9

| | Culture Bacterial Count | Colorimetric Response |
|---|---|---|
| Swab Sample | (cfu) | (Fraction Red) |
| 12A | 0 | 1.3 |
| 12B | 954 | 1.4 |
| 12C | 724 | 1.4 |
| 12D | 2089 | 1.2 |
| 12E | 6918 | 1.0 |
| 12F | 47863 | 1.0 |

### Example 13

### Comparison in the Detection Efficiency of Polydiacetylene Coated Sensors for Lysed S. aureus Versus Whole S. aureus using a Rabbit Anti Staphylococcus aureus IgG Antibody Protein Probe in PBS L64 Buffer Solution

A formulation of (60/40) diacetylene HO(O)C(CH₂)₂C(O)O(CH₂)₄C≡C—C≡C(CH₂)₄O(O)C(CH₂)₁₂CH₃ and 1,2-dimeristoyl-sn-glycero-3-phosphocholine (DMPC) prepared in Preparative Example 1 was coated onto 25 mm diameter porous polycarbonate membranes with 200 nm diameter pores (Avestin, Inc. Ottawa, Canada) to make calorimetric detector samples. The detector samples were prepared as in Preparative Example 3.

The polydiacetylene coated substrate (25 millimeter (mm) circle) was cut into four quarters. Each quarter sample was used as a sample for an experiment. The substrates were placed in separate wells of 24-well microtiter plates. Whole bacteria sample solutions were prepared by mixing 250 μl PBS L64 buffer solution containing whole S. aureus bacteria ATCC 25923 with 250 μl of antibody solution. The antibody solution contained Rabbit anti-Staphylococcus aureus (Catalog number YVS6881, Accurate Chemical and Scientific Corp.) at a concentration of 100 μg/ml in PBS L64 buffer solution. Samples containing lysed S. aureus bacteria ATCC 25923 in PBS L64 buffer solution were prepared using a lysing buffer which consisted of lysostaphin (available commercially from Sigma-Aldrich, catalog number L-4402) at a concentration of 3 micrograms/milliliter in PBS L64 buffer solution. Lysed bacteria sample solutions consisted of 250 μl of the lysed S. aureus bacteria (ATCC 25923) in PBS L64 mixed with 250 μl of the antibody solution prepared as described above. The concentration of bacteria used in the test samples varied between 0 and 10⁵ cfu/ml as reported in Table 10 below. The mixture of the bacteria and antibody solution was allowed to stand for 5 minutes and then added onto the 24-well plate containing the polydiacetylene-coated substrate. Control samples were also prepared for comparison. The control sample contained no bacteria and consisted simply of 250 μl of PBS-L64 buffer mixed with 250 μl of the antibody solution prepared as described above.

A picture was taken every 5 minutes using a digital camera. The picture was scanned using software from Adobe Systems Incorporated (San Jose, Calif.), trade designation ADOBE PHOTOSHOP version 5.0). The data in Table 10 below shows the difference in the calorimetric response between a control sample and the bacteria containing sample (either whole or lysed), measured at 15 minutes.

TABLE 10

| | Colorimetric Response | |
|---|---|---|
| | Difference from | Colorimetric Response |
| Bacteria | Control for Whole | Difference from Control |
| Concentration | Bacteria | for Lysed Bacteria |
| (cfu/ml) | (Δ Fraction Red) | (Δ Fraction Red) |
| 0 | 0 | 0 |
| 100 | 0.05 | 0.17 |
| 1,000 | 0.05 | 0.58 |
| 10,000 | 0.05 | 0.52 |
| 100,000 | 0.04 | 0.64 |

### Example 14

### Effect of Buffer Solution Composition on the Detection of Lysed S. aureus and Whole S. aureus using a Rabbit Anti Staphylococcus aureus IgG Antibody Protein Probe and Coated Polydiacetylene Sensors

Thirty-two polydiacetylene-coated substrates prepared as in Preparative Example 3 were placed at the bottom of separate wells in a 24-well microtiter plate.

The Following Sample Solutions were Prepared:

Sample 14A—500 μl of antibody in PBS L64 buffer solution as prepared in Example 2 containing either 10³ cfu/ml whole S. aureus bacteria or 10³ cfu/ml lysed S. aureus bacteria as by the lysing procedure given in Example 11.

Sample 14B—400 μl of antibody in PBS L64 buffer solution as prepared in Example 2 containing either 10³ cfu/ml whole S. aureus bacteria or 10³ cfu/ml lysed S. aureus bacteria as by the lysing procedure given in Example 11+100 μl of HEPES buffer solution.

Sample 14C—350 μl of antibody in PBS L64 buffer solution as prepared in Example 2 containing either 10³ cfu/ml whole S. aureus bacteria or 10³ cfu/ml lysed S. aureus bacteria as by the lysing procedure given in Example 11+150 μl of HEPES buffer) solution.

Sample 14D—-300111 of antibody in PBS L64 buffer solution as prepared in Example 2 containing either 10³ cfu/ml whole S. aureus bacteria or 103 cfu/ml lysed S. aureus bacteria as by the lysing procedure given in Example 11+200 μl of HEPES buffer solution.

Sample 14E—250 μl of antibody in PBS L64 buffer solution as prepared in Example 2 containing either 10³ cfu/ml whole S. aureus bacteria or 10³ cfu/ml lysed S. aureus bacteria as by the lysing procedure given in Example 11+250 μl of HEPES buffer solution.

Sample 14F—200 μl of antibody in PBS L64 buffer solution as prepared in Example 2 containing either 10³ cfu/ml whole S. aureus bacteria or 10³ cfu/ml lysed S. aureus bacteria as by the lysing procedure given in Example 11+300 μl of HEPES solution.

Sample 14G—150 μl of antibody in PBS L64 buffer solution as prepared in Example 2 containing either 10³ cfu/ml whole S. aureus bacteria or 10³ cfu/ml lysed S. aureus bacteria as by the lysing procedure given in Example 11+350 μl of HEPES buffer solution.

Sample 14H—500 μl of HEPES solution with Rabbit anti-Staphylococcus aureus (Catalog number YVS6881, Accurate Chemical and Scientific Corp.) at a concentration of 100 μg/ml and containing either 10³ cfu/ml whole S. aureus bacteria or 10³ cfu/ml lysed S. aureus bacteria as by the lysing procedure given in Example 11.

A series of control sample solutions were also prepared which were identical in composition to Samples 14A-14H except that they contained no whole or lysed bacteria.

The different sample mixtures were vortexed and allowed to stand for 5 minutes and then added to separate wells containing the polydiacetylene coated substrates. The microtiter plate was agitated on an Eberbach Model 6000 shaker (Eberbach Corp., Ann Arbor, Mich.). A picture was taken at 40 minutes using a digital camera. The picture was scanned using software from Adobe Systems Incorporated (trade designation ADOBE PHOTOSHOP version 5.0, San Jose, Calif.). The data in Table 11 below shows the difference in the colorimetric response between a control sample and the bacteria containing sample (either whole or lysed), measured at 15 minutes.

TABLE 11

| | | | | Colorimetric | Colorimetric |
|---|---|---|---|---|---|
| | | | | Response | Response |
| | | | | Difference | Difference |
| | | Volume | | from | from |
| | Volume | of | Effective | Control for | Control for |
| | of PBS | HEPES | Buffer | Whole Bacteria | Lysed Bacteria |
| | Buffer | Buffer | Ionic | @ 10³ cfu/ml | @ 10³ cfu/ml |
| Sample | Solution | Solution | Strength | (Δ Fraction | (Δ Fraction |
| Solution | (μl) | (μl) | (mM) | Red) | Red) |
| 14A | 500 | 0 | 150 | 0.2 | 0.6 |
| 14B | 400 | 150 | 121 | 0.3 | 0.8 |
| 14C | 350 | 200 | 106.5 | 0.6 | 1.1 |
| 14D | 300 | 300 | 92 | 2.0 | 2.0 |
| 14E | 250 | 250 | 77.5 | 2.1 | 1.2 |
| 14F | 200 | 200 | 63 | 0.6 | 0.9 |
| 14G | 150 | 150 | 48.5 | 0.7 | 1.0 |
| 14H | 0 | 500 | 5 | 0 | 0 |

### Example 15

### Effect of Buffer Solution Composition on the Detection of Lysed S. aureus and Whole S. aureus using a High Concentration of a Fibrinogen Protein Probe and Coated Polydiacetylene Sensors

Thirty-two polydiacetylene-coated substrates as prepared in Preparative Example 3 were placed at the bottom of separate wells in a 24-well microtiter plate.

The Following Sample Solutions were Prepared:

Sample 15A—500 μl of fibrinogen in PBS L64 buffer solution containing either 10³ cfu/ml whole S. aureus bacteria or 10³ cfu/ml lysed S. aureus bacteria as by the lysing procedure given in Example 11.

Sample 15B—400 μl of fibrinogen in PBS L64 buffer solution containing either 10³ cfu/ml whole S. aureus bacteria or 10³ cfu/ml lysed S. aureus bacteria as by the lysing procedure given in Example 11+100 μl of HEPES buffer solution.

Sample 15C—350 μl of fibrinogen in PBS L64 buffer solution containing either 10³ cfu/ml whole S. aureus bacteria or 10³ cfu/ml lysed S. aureus bacteria as by the lysing procedure given in Example 11+150 μl of HEPES buffer solution.

Sample 15D—300 μl of fibrinogen in PBS L64 buffer solution containing either 10³ cfu/ml whole S. aureus bacteria or 10³ cfu/ml lysed S. aureus bacteria as by the lysing procedure given in Example 11+200 μl of HEPES buffer solution.

Sample 15E—250 μl of fibrinogen in PBS L64 buffer solution containing either 103 cfu/ml whole S. aureus bacteria or 10³ cfu/ml lysed S. aureus bacteria as by the lysing procedure given in Example 11+250 μl of HEPES buffer solution.

Sample 15F—200 μl of fibrinogen in PBS L64 buffer solution containing either 10³ cfu/ml whole S. aureus bacteria or 10³ cfu/ml lysed S. aureus bacteria as by the lysing procedure given in Example 11+300 μl of HEPES buffer solution.

Sample 15G—150 μl of fibrinogen in PBS L64 buffer solution containing either 103 cfu/ml whole S. aureus bacteria or 10³ cfu/ml lysed S. aureus bacteria as by the lysing procedure given in Example 11+350 μl of HEPES buffer solution.

Sample 15H—500 μl of HEPES buffer solution with fibrinogen (available from Sigma, cat. No FR4129, Lot# 083K7604) at a concentration of 0.5% (w/v) and containing either 10³ cfu/ml whole S. aureus bacteria or 10³ cfu/ml lysed S. aureus bacteria as by the lysing procedure given in Example 11.

In all sample solutions 15-15H, fibrinogen was dissolved in the buffer solutions at a concentration of 0.5% (w/v). A series of control sample solutions were also prepared which were identical in composition to Samples 15A-15H except that they contained no whole or lysed bacteria.

The different sample mixtures were vortexed and allowed to stand for 5 minutes and then added to separate wells containing the polydiacetylene coated substrates. The microtiter plate was agitated on an Eberbach Model 6000 shaker (Eberbach Corp., Ann Arbor, Mich.). A picture was taken at 40 minutes using a digital camera. The picture was scanned using software from Adobe Systems Incorporated (trade designation ADOBE PHOTOSHOP version 5.0, San Jose, Calif.). Colorimetric response (CR) was determined. The data in Table 12 below shows the difference in the calorimetric response between a control sample and the bacteria containing sample (either whole or lysed), measured at 15 minutes.

TABLE 12

| | | | | Colorimetric | Colorimetric |
|---|---|---|---|---|---|
| | | | | Response | Response |
| | | | | Difference | Difference |
| | | Volume | | from | from |
| | Volume | of | Effective | Control for | Control for |
| | of PBS | HEPES | Buffer | Whole Bacteria | Lysed Bacteria |
| | Buffer | Buffer | Ionic | @ 10³ cfu/ml | @ 10³ cfu/ml |
| Sample | Solution | Solution | Strength | (Δ Fraction | (Δ Fraction |
| Solution | (μl) | (μl) | (mM) | Red) | Red) |
| 15A | 500 | 0 | 150 | −1.2 | NA |
| 15B | 400 | 150 | 121 | −0.3 | 0.3 |
| 15C | 350 | 200 | 106.5 | 1.0 | 0.5 |
| 15D | 300 | 300 | 92 | 1.4 | 0.8 |
| 15E | 250 | 250 | 77.5 | 0.4 | 0.7 |
| 15F | 200 | 200 | 63 | 0.9 | 0.4 |
| 15G | 150 | 150 | 48.5 | 0.2 | 0.4 |
| 15H | 0 | 500 | 5 | 0 | 0 |

### Example 16

### Effect of Buffer Solution Composition on the Detection of Lysed S. aureus and Whole S. aureus using a Low Concentration of a Fibrinogen Protein Probe and Coated Polydiacetylene Sensors

Thirty-two polydiacetylene-coated substrates as prepared in Preparative Example 3 were placed at the bottom of separate wells in a 24-well microtiter plate.

The Following Sample Solutions were Prepared:

Sample 16A—500 μl of fibrinogen in PBS L64 buffer solution containing either 10³ cfu/ml whole S. aureus bacteria or 10³ cfu/ml lysed S. aureus bacteria as by the lysing procedure given in Example 11.

Sample 16B—400 μl of fibrinogen in PBS L64 buffer solution containing either 10³ cfu/ml whole S. aureus bacteria or 10³ cfu/ml lysed S. aureus bacteria as by the lysing procedure given in Example 11+100 μl of HEPES buffer solution.

Sample 16C—350 μl of fibrinogen in PBS L64 buffer solution containing either 10³ cfu/ml whole S. aureus bacteria or 10³ cfu/ml lysed S. aureus bacteria as by the lysing procedure given in Example 11+150 μl of HEPES buffer solution.

Sample 16D—300 μl of fibrinogen in PBS L64 buffer solution containing either 10³ cfu/ml whole S. aureus bacteria or 10³ cfu/ml lysed S. aureus bacteria as by the lysing procedure given in Example 11+200 μl of HEPES buffer solution.

Sample 16E—250 μl of fibrinogen in PBS L64 buffer solution containing either 10³ cfu/ml whole S. aureus bacteria or 10³ cfu/ml lysed S. aureus bacteria as by the lysing procedure given in Example 11+250 μl of HEPES buffer solution.

Sample 16F—200 μl of fibrinogen in PBS L64 buffer solution containing either 10³ cfu/ml whole S. aureus bacteria or 103 cfu/ml lysed S. aureus bacteria as by the lysing procedure given in Example 11+300 μl of HEPES buffer solution.

Sample 16G—150 μl of fibrinogen in PBS L64 buffer solution containing either 10³ cfu/ml whole S. aureus bacteria or 10³ cfu/ml lysed S. aureus bacteria as by the lysing procedure given in Example 11+350 μl of HEPES buffer solution.

Sample 16H—500 μl of HEPES buffer solution with fibrinogen (available from Sigma, cat. No FR4129, Lot#083K7604) at a concentration of 0.05% (w/v) and containing either 1 cfu/ml whole S. aureus bacteria or 10³ cfu/ml lysed S. aureus bacteria as by the lysing procedure given in Example 11.

In all sample solutions 16A-16H, fibrinogen was dissolved in the buffer solutions at a concentration of 0.05% (w/v). A series of control sample solutions were also prepared which were identical in composition to Samples 16A-16H except that they contained no whole or lysed bacteria.

The different sample mixtures were vortexed and allowed to stand for 5 minutes and then added to separate wells containing the polydiacetylene coated substrates. The microtiter plate was agitated on an Eberbach Model 6000 shaker (Eberbach Corp., Ann Arbor, Mich.). A picture was taken at 40 minutes using a digital camera. The picture was scanned using software from Adobe Systems Incorporated (trade designation ADOBE PHOTOSHOP version 5.0, San Jose, Calif.). The data in Table 13 below shows the difference in the calorimetric response between a control sample and the bacteria containing sample (either whole or lysed), measured at 15 minutes.

TABLE 13

| | | | | Colorimetric | Colorimetric |
|---|---|---|---|---|---|
| | | | | Response | Response |
| | | | | Difference | Difference |
| | | Volume | | from | from |
| | Volume | of | Effective | Control for | Control for |
| | of PBS | HEPES | Buffer | Whole Bacteria | Lysed Bacteria |
| | Buffer | Buffer | Ionic | @ 10³ cfu/ml | @ 10³ cfu/ml |
| Sample | Solution | Solution | Strength | (Δ Fraction | (Δ Fraction |
| Solution | (μl) | (μl) | (mM) | Red) | Red) |
| 16A | 500 | 0 | 150 | −1.2 | NA |
| 16B | 400 | 150 | 121 | −0.6 | 0.3 |
| 16C | 350 | 200 | 106.5 | 0.5 | 0.5 |
| 16D | 300 | 300 | 92 | 1.5 | 0.8 |
| 16E | 250 | 250 | 77.5 | 1.4 | 1.1 |
| 16F | 200 | 200 | 63 | 1.2 | 0.8 |
| 16G | 150 | 150 | 48.5 | 0.5 | 0.7 |
| 16H | 0 | 500 | 5 | 0 | 0 |

### Example 17

### Detection of Methacyllin Resistant S. aureus (MRSA) using a Monoclonal Antibody Pre-Reacted with Protein A and Coated Polydiacetylene Sensors

Monoclonal IgG_{1κ} antibody against PBP2′ in MRSA cross-reacts with Protein A. This antibody was pre-reacted with Protein A and then exposed to lysed MRSA (3M Culture collection #360). The procedure for lysis of the MRSA was followed as in Example 11. The lysed MRSA was prepared in PBS L64 as described in Example 13. The concentration of bacteria lysed and used in this example were 10⁵ and 10³ cfu/ml. A control sample with no bacteria but containing only the lysis agent in PBS L64 was used. The monoclonal antibody against PBP2′ was prepared in HEPES buffer at a concentration of 100 μg/ml. The Protein A (Zymed, SanFransisco, Calif., catalog #10-1006) was also prepared in HEPES buffer at a concentration of 200 μg/ml. Two different combinations of the bacteria solution and the HEPES buffers containing the antibody and Protein A were used as described below.

Sample 17A—150 μl solution of the monoclonal antibody against PBP2′ in HEPES buffer was mixed with 100 μl of Protein A in HEPES buffer. The vial was vortexed and allowed to stand for five minutes.

Sample 17A was then mixed with 250 μl of the PBS L64 solution containing either the 10³ or 10⁵ cfu/ml or the control sample with no bacteria. The vial was vortexed and allowed to stand for 5 minutes. Three samples of PDA coated on polycarbonate membrane as described in Preparative Example 3 were placed at the bottom of a 24 well plate. The solutions with varying levels of bacteria were pipetted into separate wells. The color change from blue was followed and reported in Table 14 below.

TABLE 14

| | Bacteria | |
|---|---|---|
| Sample | concentration | |
| Solution | cfu/ml | Color @ 2 hr |
| 17A | 0 | Blue |
| 17A | 10³ | Purple |
| 17A | 10⁵ | Light Red |

Sample 17B—150 μl solution of the monoclonal antibody against PBP2′ in HEPES buffer was mixed with 50 μl of Protein A in HEPES buffer. The vial was vortexed and allowed to stand for five minutes.

Sample 17B was then mixed with 300 μl of the PBS L64 solution containing either the 10³ or 10⁵ cfu/ml or the control sample with no bacteria. The vial was vortexed and allowed to stand for 5 minutes. Three samples of PDA coated on polycarbonate membrane as described in Preparative Example 3 were placed at the bottom of a 24 well plate. The solutions with varying levels of bacteria were pipetted into separate wells. The color change from blue was followed and reported in Table 15 below.

TABLE 15

| | Bacteria | |
|---|---|---|
| Sample | concentration | Color @ 20 |
| Solution | cfu/ml | minutes |
| 17B | 0 | Red |
| 17B | 10³ | Light Red |
| 17B | 10⁵ | Blue |

### Example 18

### Detection of Methacyllin Resistant S. aureus (MRSA) using a Monoclonal Antibody as the Protein Probe and Coated Polydiacetylene Sensors

The procedure for lysis of the MRSA was followed as in Example 11. The lysed MRSA was prepared in PBS L64 as described in Example 13. The concentration of bacteria lysed and used in this example were 10⁵ and 10³ cfu/ml. A control sample with no bacteria but containing only the lysis agent in PBS L64 was also used. The monoclonal IgG_{1κ} antibody against PBP2′ was prepared in HEPES buffer at a concentration of 100 μg/ml.

The following sample solutions were then prepared:

Sample 18A—250 μl solution of the monoclonal antibody against PBP2′ in PBS L64 buffer solution was mixed with 250 μl of the PBS L64 buffer solution containing no bacteria. The vial was vortexed and allowed to stand for 5 minutes.

Sample 18B—250 μl solution of the monoclonal antibody against PBP2′ in PBS L64 buffer solution was mixed with 250 μl of the PBS L64 buffer solution containing 10³ cfu/ml lysed MRSA. The vial was vortexed and allowed to stand for 5 minutes.

Sample 18C—250 μl solution of the monoclonal antibody against PBP2′ in PBS L64 buffer solution was mixed with 250 μl of the PBS L64 buffer solution containing 10⁵ cfu/ml lysed MRSA. The vial was vortexed and allowed to stand for 5 minutes.

Three samples of PDA coated on polycarbonate membrane as described in Preparative Example 3 were placed at the bottom of a 24 well plate. The solutions with varying levels of bacteria were pipetted into separate wells, and the microtiter plate was agitated on an Eberbach Model 6000 shaker (Eberbach Corp., Ann Arbor, Mich.). A picture was taken at 45 minutes using a digital camera. The picture was scanned using software from Adobe Systems Incorporated (trade designation ADOBE PHOTOSHOP version 5.0, San Jose, Calif.). The calorimetric response for each sample is reported in Table 16 below.

TABLE 16

| | | Colorimetric |
|---|---|---|
| | Lysed bacteria | Response |
| Sample | concentration | (Fraction |
| Solution | cfu/ml | Red) |
| 18A | 0 | 2.1 |
| 18B | 10³ | 2.3 |
| 18C | 10⁵ | 3.2 |

### Example 19

### Detection of E. coli in HEPES Buffer Solution at Various Concentrations using a Polymyxin Protein Probe in HEPES Buffer Solution

Five polydiacetylene-coated substrates as prepared in Preparative Example 3 were placed at the bottom of separate wells in a 24-well microtiter plate. Polymyxin B sulfate (available commercially from Aldrich) was dissolved in HEPES buffer solution, at a concentration of 26 nanomoles/ml.

The Following Sample Solutions were Prepared:

Sample 19A—500 μl of polymyxin B sulfate in HEPES buffer solution without bacteria.

Sample 19B—500 μl of polymyxin B sulfate in HEPES buffer solution containing 10³ cfu/ml E. coli bacteria as prepared in Preparative Example 8.

Sample 19C—500 μl of polymyxin B sulfate in HEPES buffer solution containing 10⁵ cfu/ml E. coli bacteria as prepared in Preparative Example 8.

Sample 19D—500 μl of polymyxin B sulfate in HEPES buffer solution containing 10⁷ cfu/ml E. coli bacteria as prepared in Preparative Example 8.

Sample 19E—500 μl of polymyxin B sulfate in HEPES buffer solution containing 10⁹ cfu/ml E. coli bacteria as prepared in Preparative Example 8.

The different sample mixtures were vortexed and allowed to stand for 5 minutes and then added to separate wells containing the polydiacetylene coated substrates. The microtiter plate was agitated on an Eberbach Model 6000 shaker (Eberbach Corp., Ann Arbor, Mich.). A picture was taken at 30 minutes using a digital camera. The picture was scanned using software from Adobe Systems Incorporated (trade designation ADOBE PHOTOSHOP version 5.0, San Jose, Calif.). Colorimetric response (CR) was determined. The data in Table 17 below reports the colorimetric response as a function of the bacteria concentration.

TABLE 17

| | E. coli Bacteria | Colorimetric |
|---|---|---|
| | Concentration | Response |
| Sample | (cfu/ml) | (Fraction Red) |
| 19A | 0 | 2.2 |
| 19B | 1000 | 1.8 |
| 19C | 100000 | 1.2 |
| 19D | 10000000 | 0.8 |
| 19E | 1000000000 | 0.0 |

### Example 20

### Liposomes with Tricosadiynoic Acid as the Diacetylene

Liposomes with tricosadiynoic acid were made using the procedure in Preparative Example 1. Samples of (60/40) 10,12-tricosadiynoic acid/1,2-DMPC in 5 mmol 7.2 pH HEPES buffer were prepared to give 10 ml solutions that were 1 mM in diacetylene for sonification and liposome formation. Stock solutions of 10,12-tricosadiynoic acid and 1,2-DMPC were prepared separately in dichloromethane such that a 1 ml from each solution when evaporated to dryness and rehydrated in 10 ml volume of buffer gave a (60/40) 10,12-tricosadiynoic acid/1,2-DMPC complex that was 1 mM in diacetylene. The dichloromethane solutions were placed into a 6 dram vial then rotary evaporated under reduced pressure at a temperature of between 25 and 30° C. until the organic solvent was removed. The residue was further dried under high vacuum (200 mTorr) for 10 minutes to remove the last traces of solvent. The samples were rehydrated using 10 ml of HEPES buffer.

This solution was then probe sonicated using a Misonix XL202 probe sonicator (available commercially from Misonix Inc., Farmington, N.Y.). A series of sonication power levels were run on these 10 ml samples. The ranges included power levels 3, 4, 5 and 6, where power level 3 was run from 10 to 20 minutes, power level 4 from 2.5 to 7.5 minutes, power level 5 from 1 to 3 minutes and power level 6 from 1 to 2 minutes in length. Solutions were sonicated and their appearance was compared to McFarland turbidity standards for haziness. A rating of 0.5 is basically clear and 4.0 which was hazy. Solutions were sonicated to a rating between 1.0 and 2.0 on the turbidity scale. After sonication all samples were cooled to room temperature (under cover) and placed into a refrigerator at 5° C. for 20 hours for the vesicle formation.

After 20 hours several samples formed acceptable vesicles which appeared similar in size to liposomes in Preparative Example 1. These samples were then used for further investigation.

Samples that formed a grey liposome phase were coated as in Preparative Example 3. The grey phase liposomes were coated onto 200 nm polycarbonate membranes. The thickness of the coating varied from 2, 3 & 4 passes per membrane at 500 μl per pass. They were then dried in a refrigerator for 8 hours then placed in a desiccator over night.

The samples were UV exposed under a 254 nanometer wavelength lamp until they had changed to a similar blue color to those samples made in Preparative Example 2 (approximately 0.630% blue) by visual inspection. For these batches it was discovered that maximum % of blue color, approximately 0.644%, occurred after 5-7 seconds of UV exposure. This is far faster than diacetylene in Preparartive Example 2 (similar color reached in 30 seconds of exposure).

The polymerized samples were cut into four pieces. The sample pieces were placed at the bottom of the wells in a 24 well titer plate. The procedure in Example 18 was followed to create Sample solutions 20A, 20B and 20C that had antibody plus bacteria levels of 0, 1000 cfu/ml, 100,000 cfu/ml. These solutions were exposed in duplicate to the polymerized PDA coated samples. The titer plate was placed in a shaker and the shaking speed was set to 60 cycles/min.

The sample color change was monitored with time. None of the samples turned color to red. All of them remained blue even after overnight exposure.

It is thought that with adjustment of the buffer system and the use of a two buffer system for the liposome formation, the response can be adjusted to give a color change to red with this PDA system. The current system was formulated for detection with PDA of Preparative Example 1. The PDA of Example 20 is different enough in structure to influence the surface character of the vesicles and their interaction with the probe and requires a different buffer system to achieve a calorimetric response.

Various modifications and alterations to this invention will become apparent to those skilled in the art without departing from the scope and spirit of this invention. It should be understood that this invention is not intended to be unduly limited by the illustrative embodiments set forth herein and that such embodiments are presented by way of example only, with the scope of the invention intended to be limited only by the claims.

What is claimed is:

1. A colorimetric system for detecting an analyte, comprising:
a calorimetric sensor comprising:
a receptor;
a polymerized composition comprising at least one diacetylene compound;
wherein the receptor is incorporated into the polymerized composition to form a transducer; and
a buffer composition that mediates the interaction between the analyte and the transducer, wherein the buffer system comprises two or more different buffers;
wherein the transducer exhibits a color change when contacted with an analyte.

2. The calorimetric system of claim 1, wherein the buffer composition comprises two or more buffers selected from the group consisting of HEPES buffer, Imidazole buffer, PBS buffer and combinations thereof.

3. The calorimetric system of claim 1, further comprising a probe.

4. The calorimetric system of claim 1, wherein the probe is selected from the group consisting of fibrinogen, streptavidin, IgG, and combinations thereof.

5. The calorimetric system of claim 1, further comprising a surfactant.

6. The colorimetric system of claim 1, wherein the transducer is a liposome.

7. The calorimetric system of claim 1, wherein the transducer exhibits a color change upon contact with the buffer composition.

8. The calorimetric system of claim 1, wherein the buffer mediates the interaction of the analyte by ionic interactions with the transducer.

9. The calorimetric system of claim 1, wherein the buffer composition mediates the interaction of the analyte by enhancing hydrophobic interactions with the transducer.

10. The calorimetric system of claim 1, wherein the receptor comprises a phospholipid.

11. The calorimetric system of claim 10, wherein the phospholipid is selected from the group consisting of phosphocholines, phosphoethanolamines, phosphatidylethanolamines, phosphatidylserines, phosphatidylglycerols, and combinations thereof.

12. A method for the detection of an analyte, comprising:
forming a calorimetric sensor, comprising a receptor and a polymerized composition comprising a diacetylene, wherein the receptor is incorporated into the polymerized composition to form a transducer capable of exhibiting a color change;
contacting the sensor with a probe;
further contacting the sensor with a sample suspected of containing a target analyte in the presence of a buffer composition comprising two or more different buffers; and
observing a color change if the analyte is present.

13. A method for the detection of an analyte, comprising:
forming a calorimetric sensor, comprising a receptor and a polymerized composition comprising a diacetylene, wherein the receptor is incorporated into the polymerized composition to form a transducer capable of exhibiting a color change in the presence of a probe;
contacting the transducer with a sample suspected of containing a target analyte, and a probe that has an affinity for both the target analyte and the receptor in the presence of a buffer composition comprising two or more different buffers; and
observing essentially no color change if the analyte is present.

14. The method of claim 13, wherein the analyte is selected from the group consisting of S. aureus, protein A, PBP2′, E. coli, and Pseudomonas aeruginosa.

15. The method of claim 13, wherein the observable color change occurs within 60 minutes of contacting the transducer with the sample suspected of containing an analyte.
